(19) United States
(12) Patent Application Publication
(10) Pub. No.: US 2023/0051571 A1
(43) Pub. Date: Feb. 16, 2023

(54) Systems and Methods for Detection of Heart Disease

(71) Applicant: Smart Solutions IP, LLC, Wixom, MI (US)

(72) Inventor: Baljit Singh Khehra, Punjab (IN)

(73) Assignee: Smart Solutions IP, LLC, Wixom, MI (US)

(21) Appl. No.: 17/816,194

(22) Filed: Jul. 29, 2022

Related U.S. Application Data
(60) Provisional application No. 63/226,142, filed on Jul. 27, 2021.

(51) Int. Cl. G16H 50/20; G16H 20/10

(52) U.S. Cl. CPC G16H 50/20; G16H 20/10

(57) ABSTRACT

A system comprising data processing hardware, and memory hardware in communication with the data processing hardware, the memory hardware storing instructions that when executed on the data processing hardware cause the data processing hardware to perform operations comprising receiving a plurality of input data from one or more data collection devices, converting each of the plurality of input data to sub-data, converting the sub-data into a fuzzy domain by using fuzzy membership functions to create fuzzy data, performing fuzzy inference operations on the fuzzy data using a fuzzy rule base, performing deffuzification operations to convert the fuzzy data into crisp data, determining a heart disease diagnosis for a patient based on the crisp data, and transmitting data indicating a dosage amount of a medication to an administration device associated with the patient based on the heart disease diagnosis, the medication configured to treat symptoms associated with heart disease.

### CROSS REFERENCE TO RELATED APPLICATIONS

This application claims priority under 35 U.S.C. § 119(e) to U.S. Provisional Application 63/226,142, filed on Jul. 27, 2021. The disclosure of this prior application is considered part of the disclosure of this application and is hereby incorporated by reference in its entirety.

### TECHNICAL FIELD

This disclosure relates to systems and methods for detection of heart disease.

### BACKGROUND

Heart disease is one of the leading causes of death in the world. It is a disease that affects the functioning of a human heart. A number of people, often middle-aged and older, are affected by heart disease. According to the World Health Organization (WHO), approximately 23.6 million individuals will die from heart disease by 2030. One of the major reasons for these fatalities is that the risks of heart disease are either never identified or identified too late.

Typically, clinical decisions for heart disease treatment are based on the opinion of a medical expert. This process is time consuming, and the medical expert's opinion may be flawed due to human error, subjective belief, fatigue, etc. The information received from a patient to form the medical expert's opinion often involves redundant as well as interrelated symptoms and signs in medical diagnoses, especially when the patient suffers from more than one type of disease. Consequently, the medical expert may not be able to diagnose heart disease accurately. Another reason for false positive or false negative diagnoses of heart disease may be due to complex interdependence of a variety of factors. These difficulties and limitations are significant challenges for an accurate diagnosis of heart disease at an early stage. Accordingly, there remains a need to improve diagnosis accuracy of heart disease to prevent unnecessary by-pass surgery and lapsing of the optimal treatment time.

### SUMMARY

One aspect of the disclosure provides a system comprising data processing hardware and memory hardware in communication with the data processing hardware, the memory hardware storing instructions that when executed on the data processing hardware cause the data processing hardware to perform operations comprising receiving a plurality of input data from one or more data collection devices, converting each of the plurality of input data to sub-data, converting the sub-data into a fuzzy domain by using fuzzy membership functions to create fuzzy data, performing fuzzy inference operations on the fuzzy data using a fuzzy rule base, performing deffuzification operations to convert the fuzzy data into crisp data, determining a heart disease diagnosis for a patient based on the crisp data, and transmitting data indicating a dosage amount of a medication to an administration device associated with the patient based on the heart disease diagnosis, the medication configured to treat symptoms associated with heart disease.

Implementations of the disclosure may include one or more of the following optional features. In some implementations, the operations further comprise displaying the heart disease diagnosis on a display of a healthcare provider device associated with a healthcare provider supervising the patient. The healthcare provider device may be configured to be operated by the healthcare provider to modify the plurality of input data.

The one or more data collection devices may include one or more of a Holter monitor, a blood pressure monitor, a cholesterol meter or monitor, a blood glucose meter or monitor, an electrocardiograph (ECG or EKG) monitor or machine, a heart rate monitor, an exercise or activity monitor, a gamma camera, a fluoroscope, a scale, an X-ray machine, a spirometer, a pulse oximeter, a capnography monitor, or a blood test machine.

The crisp data may include a numerical value indicating on a scale the likelihood that the patient has heart disease.

The input data may include one or more of chest pain, blood pressure (systolic, diastolic), cholesterol, blood sugar, resting electrocardiography, maximum heart rate, exercise, old peak, sex, thallium scan, age, slope (slope of peak exercise ST segment), color vessels (number of major vessels colored by fluoroscopy), body mass index (BMI), narrow vessels, FEV1/FVC ratio (Tiffeneau-Pinelli index), blood oxygen level, respiratory status, or c-reactive protein (CRP).

The deffuzification operations may include one or more of a Weighted Average Formulate (WAF) Method, a Quality Method (QM), a Maximum-Weighted Average Formula (MAX-WAF) Method, or a Center of Sums (COS) Method.

Another aspect of the disclosure provides a method comprising receiving, via data processing hardware, a plurality of input data from one or more data collection devices, converting, via the data processing hardware, each of the plurality of input data to sub-data, converting, via the data processing hardware, the sub-data into a fuzzy domain by using fuzzy membership functions to create fuzzy data, performing, via the data processing hardware, fuzzy inference operations on the fuzzy data using a fuzzy rule base, performing, via the data processing hardware, deffuzification operations to convert the fuzzy data into crisp data, determining, via the data processing hardware, a heart disease diagnosis for a patient based on the crisp data, and transmitting, via the data processing hardware, data indicating a dosage amount of a medication to an administration device associated with the patient based on the heart disease diagnosis, the medication configured to treat symptoms associated with heart disease. This aspect may include one or more of the following optional features.

In some implementations, the method includes displaying, via the data processing hardware, the heart disease diagnosis on a display of a healthcare provider device associated with a healthcare provider supervising the patient. The healthcare provider device may be configured to be operated by the healthcare provider to modify the plurality of input data.

The one or more data collection devices may include one or more of a Holter monitor, a blood pressure monitor, a cholesterol meter or monitor, a blood glucose meter or monitor, an electrocardiograph (ECG or EKG) monitor or machine, a heart rate monitor, an exercise or activity monitor, a gamma camera, a fluoroscope, a scale, an X-ray machine, a spirometer, a pulse oximeter, a capnography monitor, or a blood test machine.

The crisp data may include a numerical value indicating on a scale the likelihood that the patient has heart disease.

The input data may include one or more of chest pain, blood pressure (systolic, diastolic), cholesterol, blood sugar, resting electrocardiography, maximum heart rate, exercise, old peak, sex, thallium scan, age, slope (slope of peak exercise ST segment), color vessels (number of major vessels colored by fluoroscopy), body mass index (BMI), narrow vessels, FEV1/FVC ratio (Tiffeneau-Pinelli index), blood oxygen level, respiratory status, or c-reactive protein (CRP).

The deffuzification operations may include one or more of a Weighted Average Formulate (WAF) Method, a Quality Method (QM), a Maximum-Weighted Average Formula (MAX-WAF) Method, or a Center of Sums (COS) Method.

Another aspect of the disclosure provides a system comprising data processing hardware and memory hardware in communication with the data processing hardware, the memory hardware storing instructions that when executed on the data processing hardware cause the data processing hardware to perform operations comprising receiving a plurality of input data from the one or more data collection devices, converting each of the plurality of input data to sub-data, converting the sub-data into a fuzzy domain by using fuzzy membership functions to create fuzzy data, performing fuzzy inference operations on the fuzzy data using a fuzzy rule base, performing deffuzification operations to convert the fuzzy data into crisp data, determining a heart disease diagnosis for a patient based on the crisp data, and transmitting data indicating a dosage amount of a medication to an administration device associated with the patient based on the heart disease diagnosis, the medication configured to treat symptoms associated with heart disease. This aspect may include one or more of the following optional features.

In some implementations, the operations include displaying the heart disease diagnosis on a display of a healthcare provider device associated with a healthcare provider supervising the patient. The healthcare provider device may be configured to be operated by the healthcare provider to modify the plurality of input data.

The one or more data collection devices may include one or more of a Holter monitor, a blood pressure monitor, a cholesterol meter or monitor, a blood glucose meter or monitor, an electrocardiograph (ECG or EKG) monitor or machine, a heart rate monitor, an exercise or activity monitor, a gamma camera, a fluoroscope, a scale, an X-ray machine, a spirometer, a pulse oximeter, a capnography monitor, or a blood test machine.

The crisp data may include a numerical value indicating on a scale the likelihood that the patient has heart disease.

The input data may include one or more of chest pain, blood pressure (systolic, diastolic), cholesterol, blood sugar, resting electrocardiography, maximum heart rate, exercise, old peak, sex, thallium scan, age, slope (slope of peak exercise ST segment), color vessels (number of major vessels colored by fluoroscopy), body mass index (BMI), narrow vessels, FEV1/FVC ratio (Tiffeneau-Pinelli index), blood oxygen level, respiratory status, or c-reactive protein (CRP).

The deffuzification operations may include one or more of a Weighted Average Formulate (WAF) Method, a Quality Method (QM), a Maximum-Weighted Average Formula (MAX-WAF) Method, or a Center of Sums (COS) Method.

The details of one or more implementations of the disclosure are set forth in the accompanying drawings and the description below. Other aspects, features, and advantages will be apparent from the description and drawings, and from the claims.

### DESCRIPTION OF DRAWINGS

Reference will now be made to the accompanying Figures, which are not necessarily drawn to scale, and wherein:

FIG. 1 is a schematic view of a system for determining a heart disease diagnosis in accordance with an exemplary embodiment of the present disclosure;

FIG. 2 is a schematic view of a heart disease diagnosis application of the system of FIG. 1;

FIG. 3A is a graphical representation of a membership function of an input variable “Chest Pain”;

FIG. 3B is a graphical representation of a membership function of an input variable “Blood Pressure-Systolic”;

FIG. 3C is a graphical representation of a membership function of an input variable “Blood Pressure-Diastolic”;

FIG. 3D is a graphical representation of a membership function of an input variable “Cholesterol”;

FIG. 3E is a graphical representation of a membership function of an input variable “Blood Sugar (Diabetes)”;

FIG. 3F is a graphical representation of a membership function of an input variable “Resting Electrocardiography (ECG)”;

FIG. 3G is a graphical representation of a membership function of an input variable “Maximum Heart Rate”;

FIG. 3H is a graphical representation of a membership function of an input variable “Exercise”;

FIG. 3J is a graphical representation of a membership function of an input variable “Old Peak”;

FIG. 3K is a graphical representation of a membership function of an input variable “Thallium Scan”;

FIG. 3M is a graphical representation of a membership function of an input variable “Sex”;

FIG. 3N is a graphical representation of a membership function of an input variable “Age”;

FIG. 3P is a graphical representation of a membership function of an input variable “Slope”;

FIG. 3Q is a graphical representation of a membership function of an input variable “Color Vessels”;

FIG. 3R is a graphical representation of a membership function of an input variable “BMI”;

FIG. 3S is a graphical representation of a membership function of an input variable “Narrow Vessels”;

FIG. 3T is a graphical representation of a membership function of an input variable “FEV1/FVC Ratio”;

FIG. 3U is a graphical representation of a membership function of an input variable “Blood Oxygen Level”;

FIG. 3V is a graphical representation of a membership function of an input variable “Respiratory Status”;

FIG. 3W is a graphical representation of a membership function of an input variable “CRP”;

FIG. 4 is a graphical representation of a membership function of an output variable “Heart Disease”;

FIG. 5A is a Table 1 showing membership rules;

FIG. 5B is a continuation of Table 1 of FIG. 5A showing membership rules;

FIG. 5C is a continuation of Table 1 of FIG. 5B showing membership rules;

FIG. 6 is an exemplary graphical user interface (GUI) displayed on a healthcare provider (HCP) system in accordance with an exemplary embodiment of the present disclosure;

FIG. 7 is a flowchart illustrating an exemplary method for determining a heart disease diagnosis in accordance with an exemplary embodiment of the present disclosure; and

FIG. 8 is a schematic view of an exemplary computing device that may be used to implement the systems and methods described herein in accordance with an exemplary embodiment of the present disclosure.

Like reference symbols in the various drawings indicate like elements.

### DETAILED DESCRIPTION

Some implementations of the disclosed technology will be described more fully with reference to the accompanying drawings. This disclosed technology may, however, be embodied in many different forms and should not be construed as limited to the implementations set forth herein.

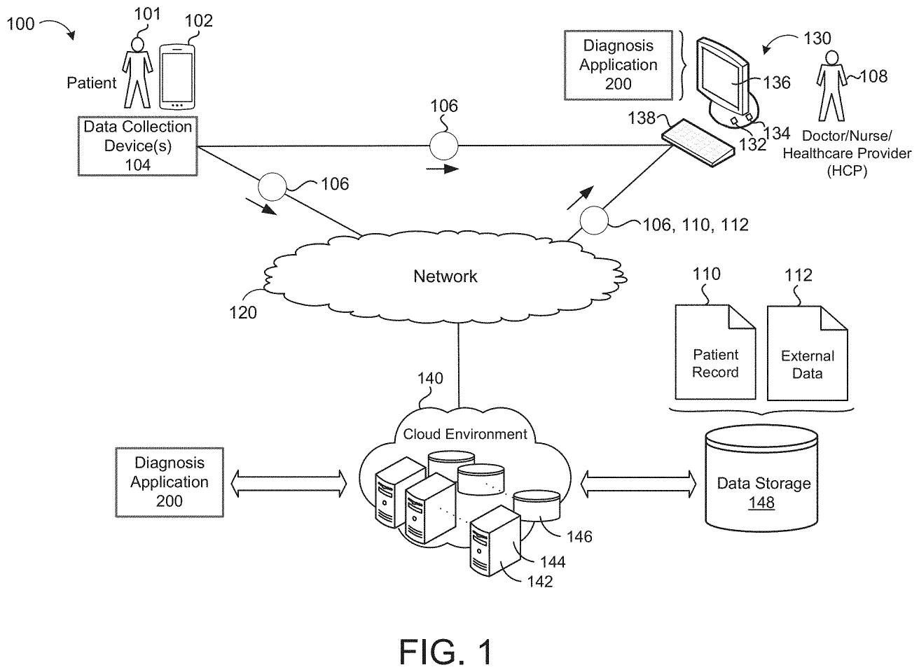

Referring to FIG. 1, in some implementations, a heart disease diagnosis system 100 is generally shown. As set forth in greater detail below, appropriate data collection and processing approaches (e.g., Information Technology (IT)) may provide improvements in heart disease diagnoses. After collection of appropriate data of the patient, Artificial Intelligence (AI) techniques may be applied for heart disease diagnosis. For example, soft-computing-based AI techniques may be applied to the heart disease diagnosis system 100. Soft computing aims to exploit tolerances for uncertainty, imprecision, and partial truth. From clinical data, vagueness, impreciseness, and uncertainty may be important aspects of heart disease symptoms. Fuzzy logic is an exemplary soft computing approach that may be applied to handle incomplete, imprecise, fragmentary, ambiguous, partially-reliable, vague, and/or contradictory information. Accordingly, in some implementations, a fuzzy logic system may be implemented to develop heart disease diagnosis system 100 for predicting the risk levels of heart disease.

The heart disease diagnosis system 100 may include one or more data collection devices 104, a network 120, a healthcare provider (HCP) system 130 associated with an authorized HCP 108, and a heart disease diagnosis application 200. The one or more data collection devices 104 are configured to obtain data 106 associated with a patient 101. For example, some of the data collection devices 104 may obtain the data 106 via active collection, i.e., the patient 101 actively inputting the data 106 directly into the data collection device 104. Such data may include the patient's age, sex, etc. As another example, some of the data collection devices 104 may obtain the data 106 via passive collection, i.e., the data collection device 104 may passively obtain the data 106 from the patient 101, e.g., via sensors, monitors, etc. Such data may include the patient's heart rate, blood pressure, blood oxygen level, etc. As yet another example, the data 106 may be collected through an intermediate device, person, etc. For example, the patient 101 may provide the HCP 108 with the data 106 by, e.g., directly telling the HCP 108, sending the data 106 to the HCP system 130 associated with the HCP 108 via an email or other data transmission process, any other suitable means for the patient 101 conveying the data 106 to the HCP 108 or the HCP system 130. In other implementations, the system may include a patient device 102 (e.g., a smartphone, desktop computer, laptop computer, tablet computer, smartwatch, etc.) associated with the patient 101 that is configured to receive the data 106 from the data collection devices 104 and transmit the data 106 to the HCP system 130.

The data collection devices 104 may include, but are not limited to, a Holter monitor, a blood pressure monitor, a cholesterol meter or monitor, a blood glucose meter or monitor, an electrocardiograph (ECG or EKG) monitor or machine, a heart rate monitor, an exercise or activity monitor, a gamma camera, a fluoroscope, a scale, an X-ray machine, a spirometer, a pulse oximeter, a capnography monitor, a blood test machine, etc. As set forth above, the data collection devices 104 are configured to transmit the data 106 to the HCP system 130. Such data transmission may be via a wired or wireless connection.

The system 100 may include an administration device 103 associated with the patient 101. The administration device 103 may include a doser 103a and an administration computing device 103b in communication with the doser 103a. The administration computing device 103b may be in communication with the patient device 102, the data collection devices 104, and/or the HCP system 130, e.g., via the network 120. The administration computing device 103b may be configured to receive a dose of a medication as part of a treatment method for the patient 101 to treat heart disease, prevent heart disease from developing, and/or treat symptoms associated with heart disease, and the doser 103a may be configured to administer the dose of medication to the patient 101. The administration device 103 may include an injection unit, a smart pen, a smart pill bottle/cap, a smart pill, an infusion pump, etc.

The medication may include, but is not limited to, quinapril, perindopril, nifedipine, ramipril, hydralazine, hydrochlorothiazide and olmesartan, ticagrelor, amlodipine and atorvastatin, captopril, carveilol, losartan, isosorbide dinitrate, valsartan, prasugrel, isosorbide mononitrate, eplerenone, metoprolol, benazepril, amlodipine and benazepril, enoxaparin, trandolapril, fosinopril, nesiritide, amlodipine, clopidogrel, lisinopril, moexipril, enalapril, and bisoprolol.

The HCP 108 supervising the patient 101 may operate the HCP system 130 to execute the diagnosis application 200. The HCP 108 may include a physician, nurse, clinician, or other qualified health professionals.

The network 120 may include any type of network that allows sending and receiving communication signals, such as a wireless telecommunication network, a cellular telephone network, a time division multiple access (TDMA) network, a code division multiple access (CDMA) network, Global system for mobile communications (GSM), a third generation (3G) network, fourth generation (4G) network, fifth generation (5G) network, sixth generation (6G) network, a satellite communications network, and other communication networks. The network 120 may include one or more of a Wide Area Network (WAN), a Local Area Network (LAN), and a Personal Area Network (PAN). In some examples, the network 120 includes a combination of data networks, telecommunication networks, and a combination of data and telecommunication networks. The data collection devices 104, the HCP system 130, and, in some implementations, the patient device 102 communicate with each other by sending and receiving signals (wired or wireless) via the network 120, which, in some examples, may utilize Bluetooth, Wi-Fi, etc. In some examples, the network 120 provides access to cloud computing resources 140, which may be elastic/on-demand computing and/or storage resources 146 available over the network 120. The term “cloud” services generally refers to a service performed not locally on a user's device, but rather delivered from one or more remote devices accessible via one or more networks 120.

The patient device 102 may include, but is not limited to, a portable electronic device (e.g., smartphone, cellular phone, personal digital assistant, personal computer, wireless tablet device, or a wearable device such as a smartwatch, etc.), a desktop computer, or any other electronic device capable of sending and receiving information via the network 120. The patient device 102 may include data processing hardware (a computing device that executes instructions), memory hardware, and a display in communication with the data processing hardware. In some examples, the patient device 102 may include a keyboard, mouse, microphones, and/or a camera for allowing the patient 101 to input data. In addition to or in lieu of the display, the patient device 102 may include one or more speakers to output audio data to the patient 101. In some implementations, the patient device 102 may include one or more of the data collection devices 104. For example, the patient device 102 may be connected (e.g., wired or wirelessly) to one or more sensors configured to generate the data 106 associated with the patient 101. For example, the one or more sensors may include a heart rate monitor (pulse/EKG), an accelerometer, a hydration sensor, a glucose sensor, a blood pressure sensor, a temperature sensor, a weight sensor, etc.

The storage resources 146 may provide data storage 148 for storing a patient record 110 associated with the patient 101 and external data 112 associated with the patient 101. The patient record 110 may be encrypted while stored on the data storage 148 so that any information identifying patient 101 is anonymized, but may later be decrypted when the patient 101 or supervising HCP 108 requests the patient record 110 (assuming the requester is authorized/authenticated to access the patient record 110). All data transmitted over the network 120 between the data collection devices 104, the HCP system 130, in some implementations, the patient device 102, and the cloud computing system 140 may be encrypted and sent over secure communication channels.

The external data 112 may include the data 106 that is collected from the data collection devices 104 or other suitable data collection devices. For example, one of the data collection devices 104 may obtain the data 106 and transmit it to the data storage 148 where it is stored as external data 112. In other implementations, one or more tests may be performed on the patient 101 at a remote location to obtain the external data 112, and the external data 112 is then entered and stored in the data storage 148 in any suitable manner, e.g., being input by the patient 101 (i.e., via the patient device 102), being input by the HCP 108, or being transmitted from a remote machine or device to the data storage 148. That is, the data used by the diagnosis application 200 may be sourced from the data 106 from the data collection devices 104 and the external data 112.

The HCP system 130 may be located at a clinic, doctor's office, or facility administered by the HCP 108 and includes data processing hardware 132, memory hardware 134, and a display 136. The memory hardware 134 and the display 136 are in communication with the data processing hardware 132. For instance, the data processing hardware 132 may reside on a desktop computer or portable electronic device for allowing the HCP 108 to input and retrieve data to and from the diagnosis application 200. In some examples, the HCP 108 may onboard some or all of patient data 106 to the diagnosis application 200. The HCP system 130 includes a keyboard 138, mouse, microphones, speakers and/or a camera.

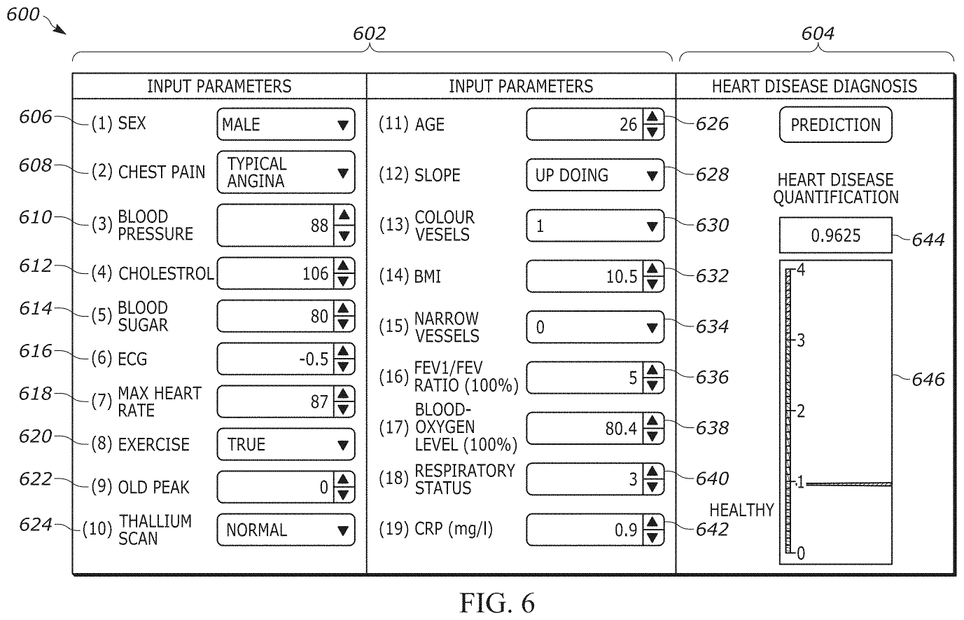

In some implementations, the HCP system 130 (i.e., via the data processing hardware 132) executes the diagnosis application 200 (or accesses a web-based patient application) for establishing a connection with the diagnosis application 200 to input and retrieve data therefrom. For example, all or a portion of the diagnosis application 200 may be executed locally on the HCP system 130. As another example, all or a portion of the diagnosis application 200 may be executed by the cloud resources 140, and the HCP system 130 may execute a portal or local application for accessing the diagnosis application 200 in the cloud. The diagnosis application 200, when executing on the data processing hardware 132 of the HCP system 130, is configured to display a variety of graphical user interfaces (GUIs) (e.g., a heart disease diagnosis GUI 600 as shown in FIG. 6) on the display 136 of the HCP system 130 that, among other things, allow the HCP 108 to input the data 106 corresponding to the patient 101.

The cloud computing resources 140 may be a distributed system (e.g., remote environment) having scalable/elastic resources 142. The resources 142 include computing resources 144 (e.g., data processing hardware) and/or the storage resources 146 (e.g., memory hardware). As set forth above, in some implementations, the cloud computing resources 140 execute all or a portion of the diagnosis application 200 for facilitating communications with the patient device 102 and the HCP system 130 and storing data on the storage resources 146 within the data storage 148.

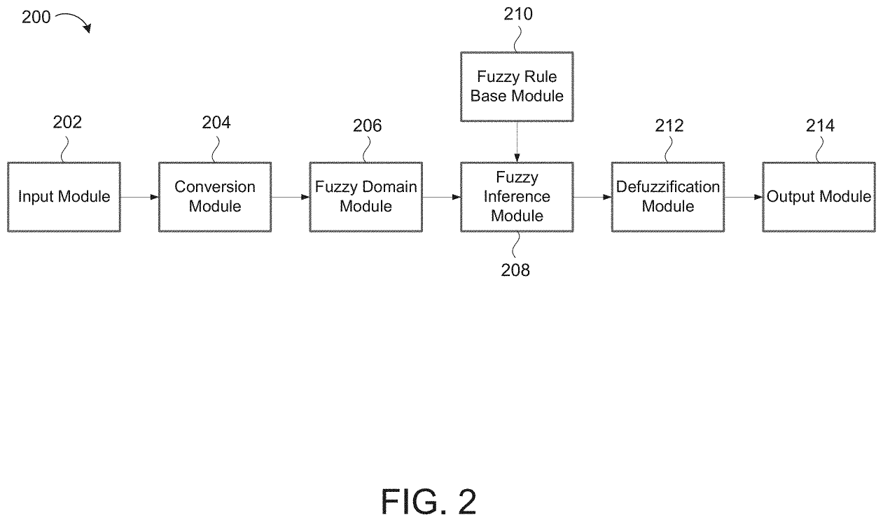

Referring to FIG. 2, a diagram illustrating the heart disease diagnosis application 200 is generally shown. According to one example, aspects of the application 200 may be executed by the computing resources 144 of the cloud computing system 140. In another example, aspects of the diagnosis application 200 may be executed by an electronic device, such as the data processing hardware 132 of the HCP system 130. In another example, aspects of the diagnosis application 200 may be executed by some combination of the computing resources 144 and the data processing hardware 132. The diagnosis application 200 includes an input module 202, a conversion module 204, a fuzzy domain module 206, a fuzzy inference module 208, a fuzzy rule base module 210, a defuzzification module 212, and an output module 214.

The input module 202 is configured to obtain (i.e., fetch or receive) input data 216 associated with the patient 102, the input data 216 including the data 106, the patient record 110, and the external data 112. For example, the input module 202 is configured to obtain the data 106 from the data collection devices 104 and/or from the patient device 102. The input module 202 is also configured to obtain (i.e., fetch or receive) the patient record 110 and the external data 112 from the data storage 148. The input data 216 includes, but is not limited to, the patient's chest pain, blood pressure (systolic, diastolic), cholesterol, blood sugar, resting electrocardiography, maximum heart rate, exercise, old peak, sex, thallium scan, age, slope (slope of peak exercise ST segment), color vessels (number of major vessels colored by fluoroscopy), body mass index (BMI), narrow vessels, FEV1/FVC ratio (Tiffeneau-Pinelli index), blood oxygen level, respiratory status, c-reactive protein (CRP), etc. The input module 202 is configured to transmit the input data 216 to the conversion module 204.

The conversion module 204 is configured to receive the input data 216 from the input module 202. The conversion module 204 is configured to convert or divide the input data 216 to one of a plurality of sub-data points or sub-variables 218. That is, the conversion module 204 is configured to convert each individual data point of the input data 216 to one sub-variable 218 of a plurality of sub-variables 218 for that data point.

As one example, the conversion module 204 may convert the input data 216 including chest pain to one of a plurality of sub-variables 218 including typical angina, atypical angina, non-angina pain, and asymptomatic.

As another example, the conversion module 204 may convert the input data 216 including blood pressure (systolic) to one of a plurality of sub-variables 218 including low, medium, high, and very high. As another example, the conversion module 204 may convert the input data 216 including blood pressure (diastolic) to one of a plurality of sub-variables 218 including low blood pressure, normal blood pressure, hypertension stage I, hypertension stage II, and hypertensive crisis. The diastolic blood pressure input data 216 may be defined as blood pressure between 30 and 150. Low diastolic blood pressure may be between 30 and 65, normal diastolic blood pressure may be between 65 and 90, hypertension stage I may be between 90 and 100, hypertension stage II may be between 100 and 110, and hypertensive crisis may be greater than 110.

As another example, the conversion module 204 may convert the input data 216 including cholesterol to one of a plurality of sub-variables 218 including low cholesterol, medium cholesterol, high cholesterol, and very high cholesterol.

As another example, the conversion module 204 may convert the input data 216 including blood sugar to one of a plurality of sub-variables 218 including B S True.

As another example, the conversion module 204 may convert the input data 216 including resting electrocardiography to one of a plurality of sub-variables 218 including normal resting electrocardiography 1, ST-T abnormal, and hypertrophy.

As another example, the conversion module 204 may convert the input data 216 including maximum heart rate to one of a plurality of sub-variables 218 including low maximum heart rate, medium maximum heart rate, and high maximum heart rate.

As another example, the conversion module 204 may convert the input data 216 including exercise to one of a plurality of sub-variables 218 including exercise true.

As another example, the conversion module 204 may convert the input data 216 including old peak to one of a plurality of sub-variables 218 including low old peak, risk old peak, and terrible old peak.

As another example, the conversion module 204 may convert the input data 216 including thallium scan to one of a plurality of sub-variables 218 including normal thallium scan, fixed defect thallium scan, and reversible defect thallium scan.

As another example, the conversion module 204 may convert the input data 216 including sex to one of a plurality of sub-variables 218 including male and female.

As another example, the conversion module 204 may convert the input data 216 including age to one of a plurality of sub-variables 218 including young age, mild age, old age, and very old age.

As another example, the conversion module 204 may convert the input data 216 including slope (slope of peak exercise ST segment) to one of a plurality of sub-variables 218 including up-sloping, flat, and down-sloping.

As another example, the conversion module 204 may convert the input data 216 including color vessels (number of major vessels colored by fluoroscopy) to one of a plurality of sub-variables 218 including zero colored vessels, one colored vessel, two colored vessels, and three colored vessels.

As another example, the conversion module 204 may convert the input data 216 including body mass index (BMI) to one of a plurality of sub-variables 218 including underweight, normal, overweight, and obese. Underweight may be less than 18.5, normal BMI may be between 18.5 and 24.9, overweight may be between 25 and 29.9, and obese may be equal to or greater than 30.

As another example, the conversion module 204 may convert the input data 216 including narrow vessels (Angiographic Disease status: number of major vessels with >50% narrowing) to one of a plurality of sub-variables 218 including zero narrow vessels, one narrow vessel, two narrow vessels, three narrow vessels, and four narrow vessels.

As another example, the conversion module 204 may convert the input data 216 including FEV1/FVC ratio to one of a plurality of sub-variables 218 including severe, moderate, mild, and normal. Normal FEV1/FVC ratio may be equal to or greater than 80%, mild FEV1/FVC ratio may be between 60% and 79%, moderate FEV1/FVC ratio may be between 40% and 59%, and severe FEV1/FVC ratio may be less than 40%.

As another example, the conversion module 204 may convert the input data 216 including blood oxygen level (oxygen saturation (SpO2)) to one of a plurality of sub-variables 218 including normal blood oxygen level, mild blood oxygen level, and low blood oxygen level. Normal blood oxygen level may be between 95% and 100%, mild blood oxygen level may be between 90% and 95%, and low blood oxygen level may be less than 90%.

As another example, the conversion module 204 may convert the input data 216 including respiratory status to one of a plurality of sub-variables 218 including normal respiratory status, attention respiratory status, intervention respiratory status, immediate intervention respiratory status. The respiratory status may combine four respiratory measurements into a single number on a scale from 1 to 10. This scale may be referred to as the Integrated Pulmonary Index algorithm (IPI). The IPI includes measurements for the level of carbon dioxide in the lungs, pulse oxygenation, pulse rate, and respiration rate. Normal respiratory status may be between 8 and 10, attention respiratory status may be between 5 and 7, intervention respiratory status may be between 3 and 4, and immediate intervention respiratory status may be between 1 and 2.

As another example, the conversion module 204 may convert the input data 216 including c-reactive protein (CRP) to one of a plurality of sub-variables 218 including low risk, intermediate risk, and high risk. Low risk may be less than 1.0 mg, intermediate risk may be between 1.0 mg and 2.9 mg, and high risk may be greater than 3.0 mg.

The conversion module 204 is configured to transmit the sub-variables 218 to the fuzzy domain module 206, which is configured to receive the sub-variables 218 from the conversion module 204. The fuzzy domain module 206 is configured to convert the sub-variables 218 into a fuzzy domain using fuzzy membership functions. In some implementations, the fuzzy domain module 2016 may implement trapezoidal and triangular fuzzy membership functions to convert the sub-variables 218 into the fuzzy domain.

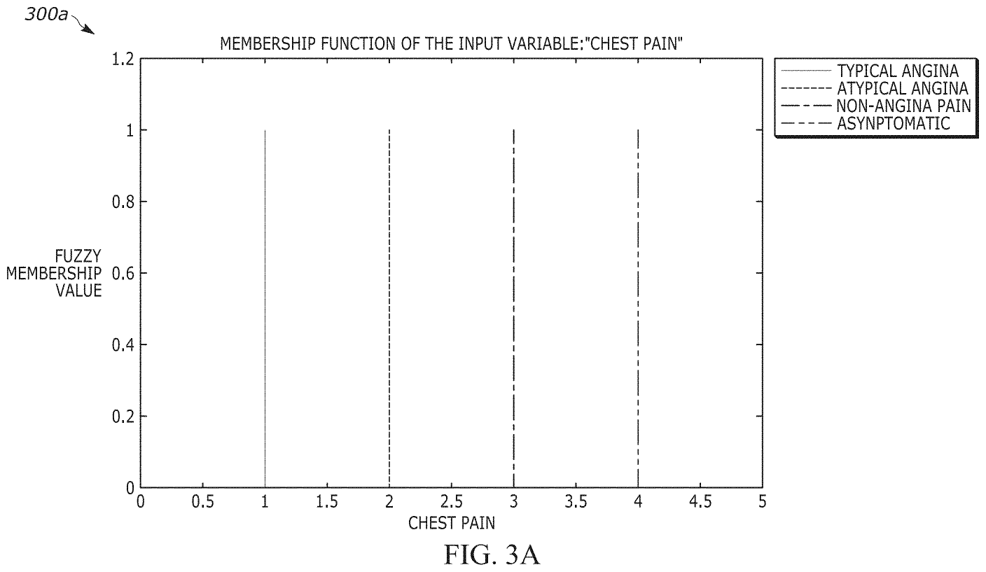

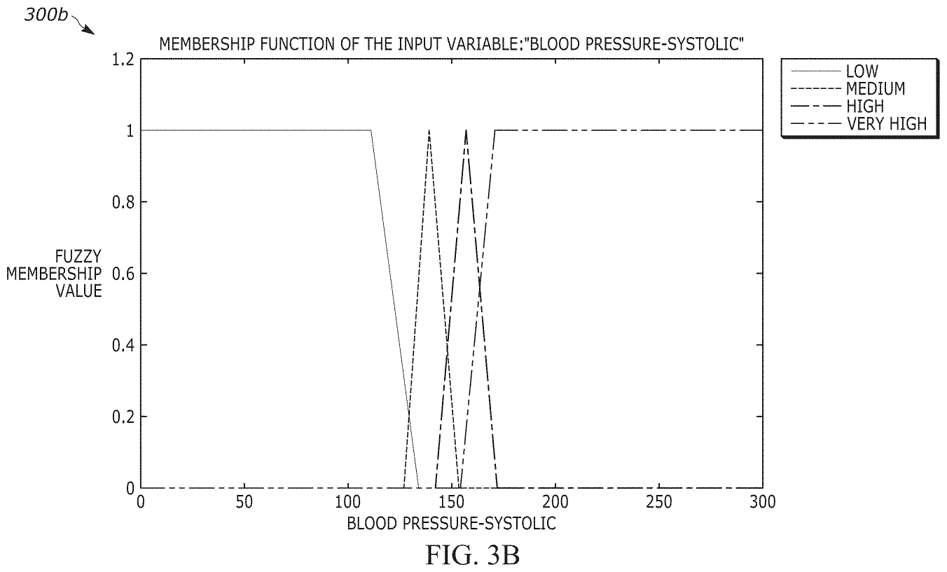

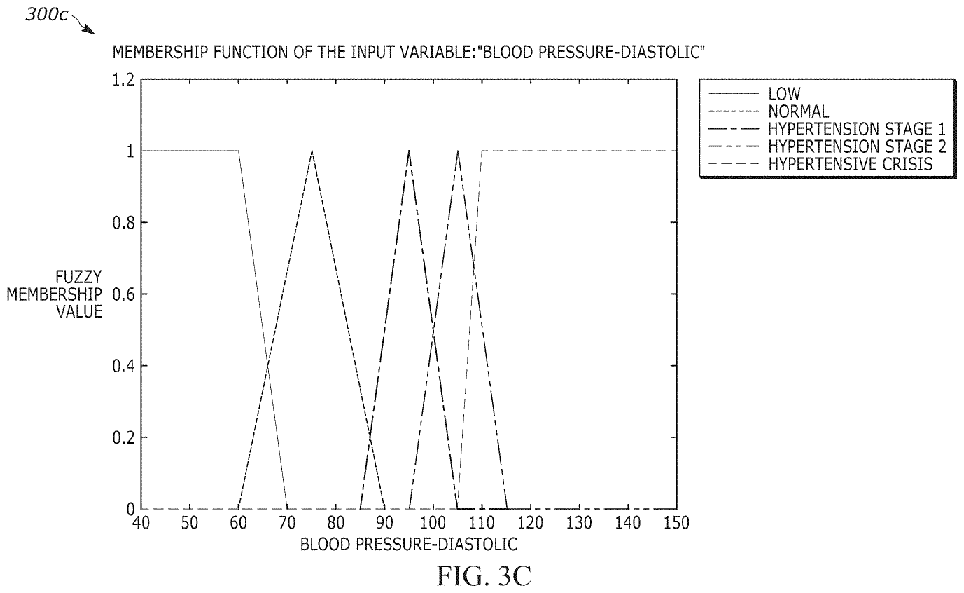

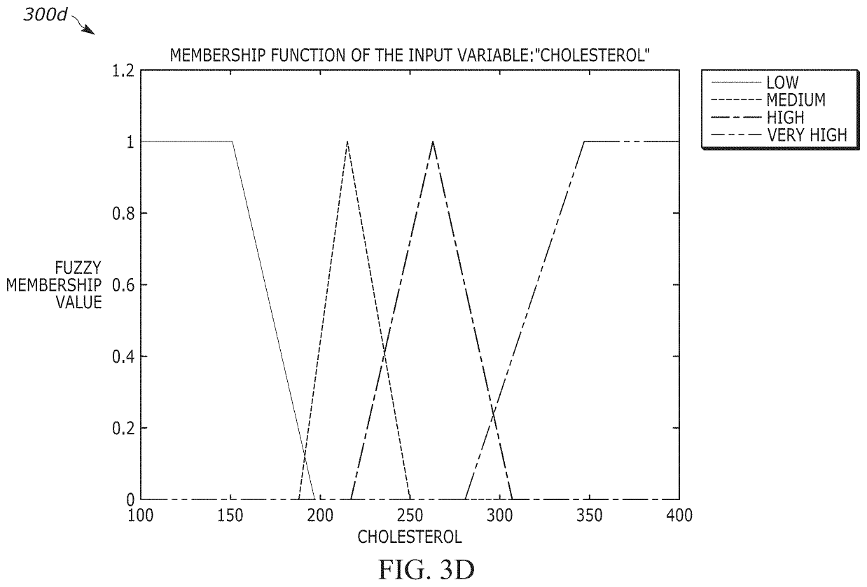

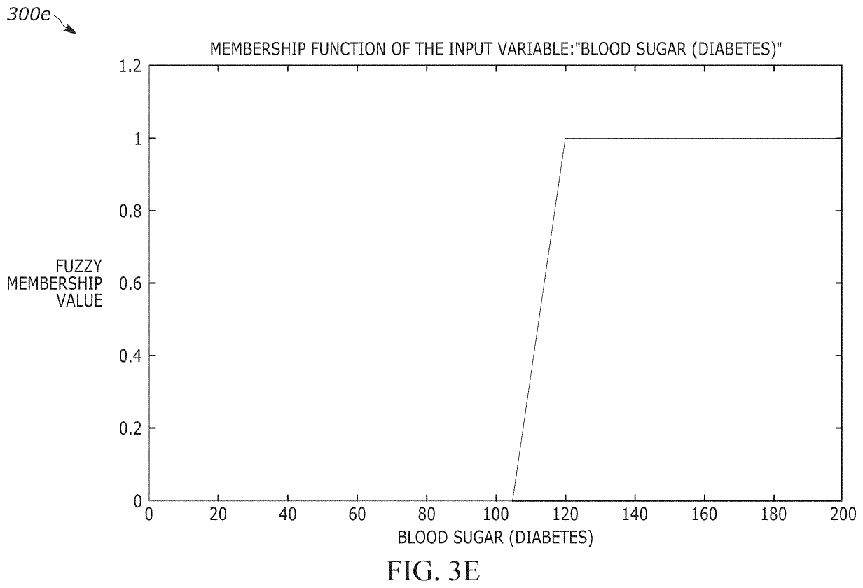

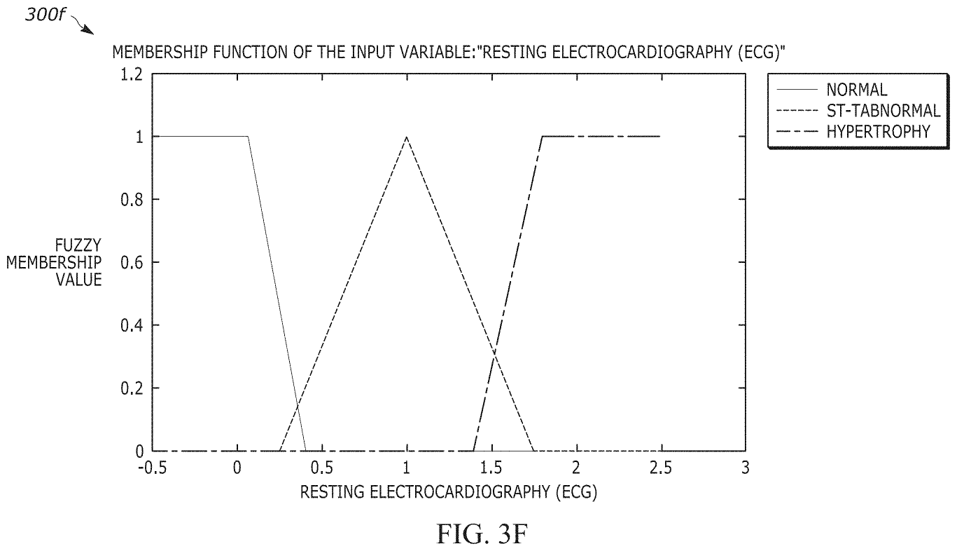

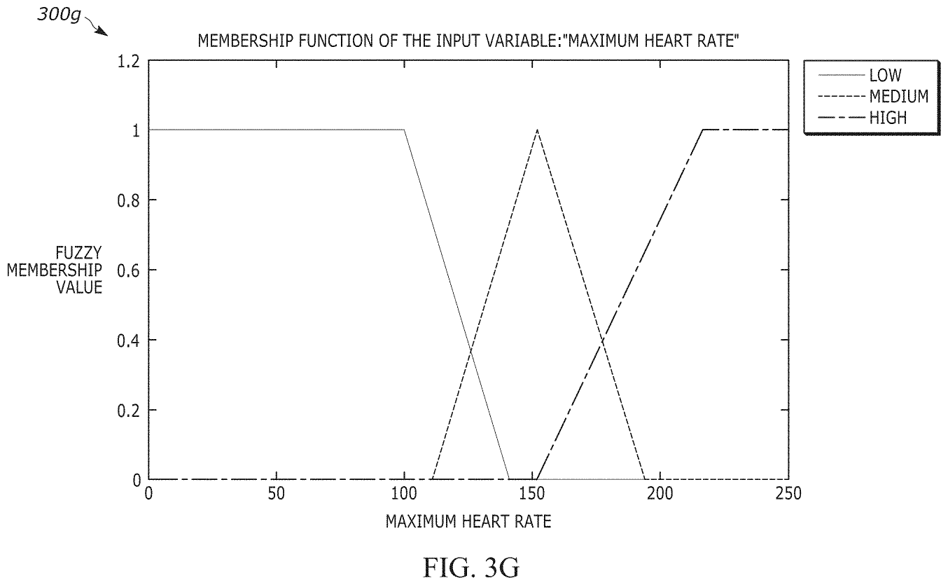

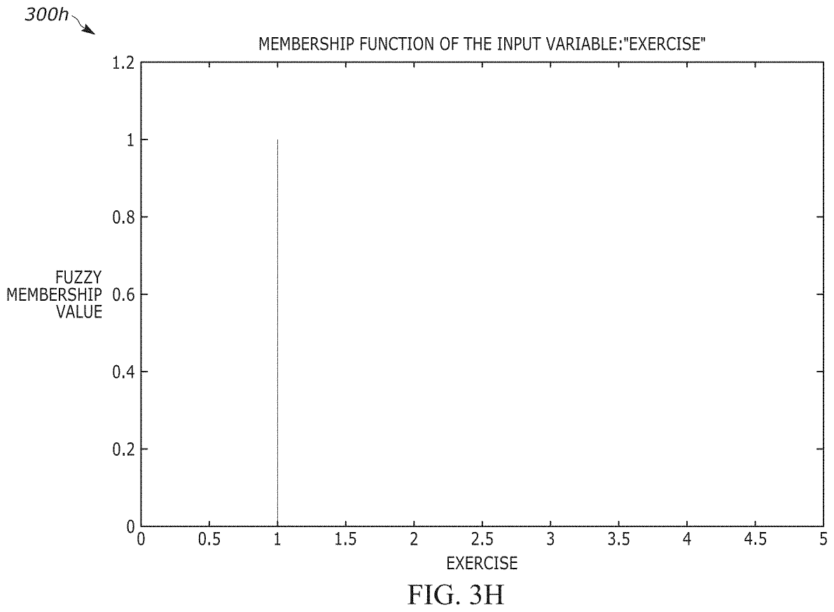

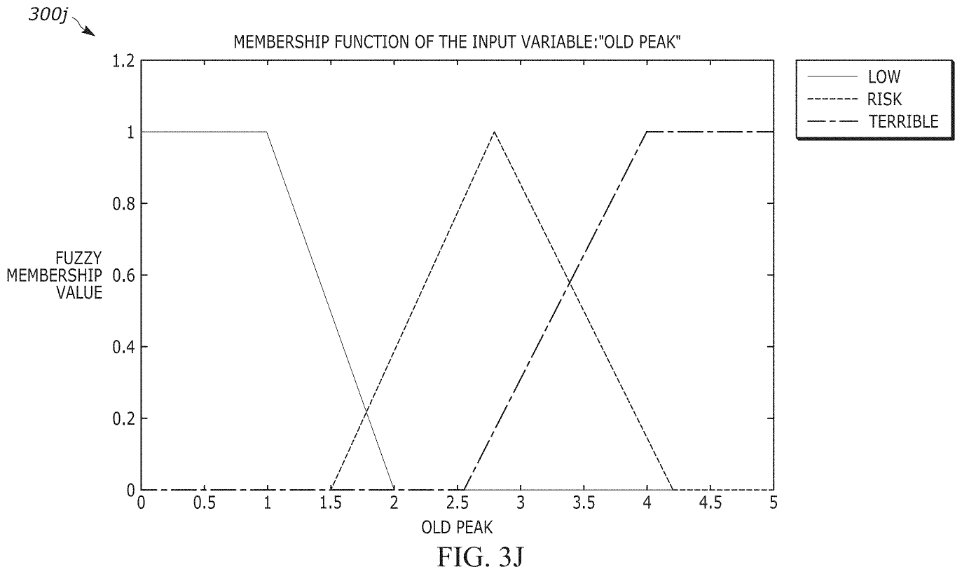

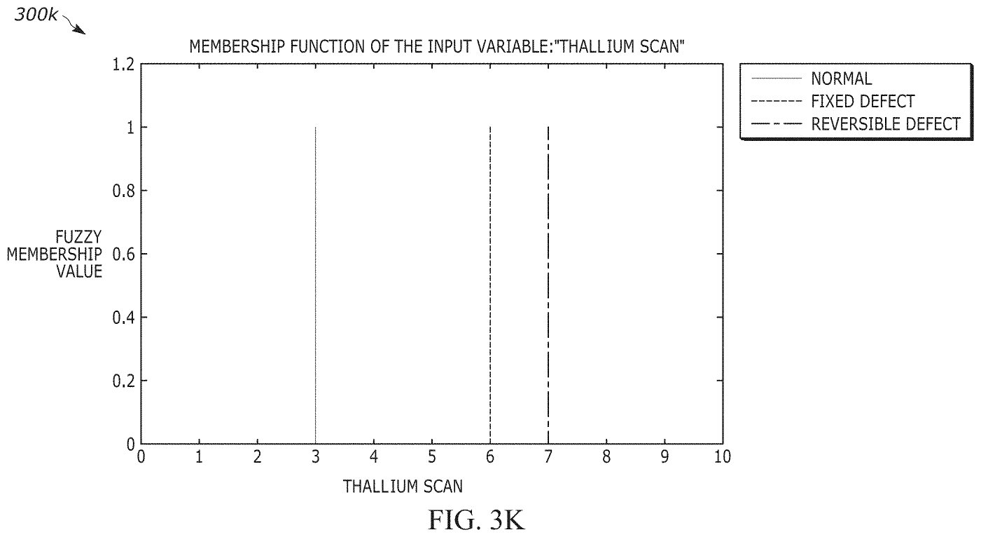

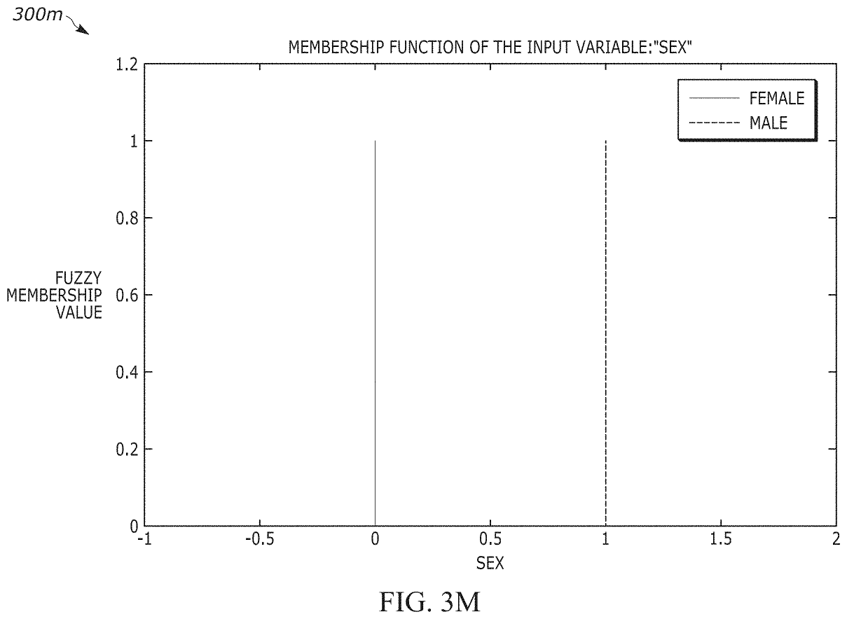

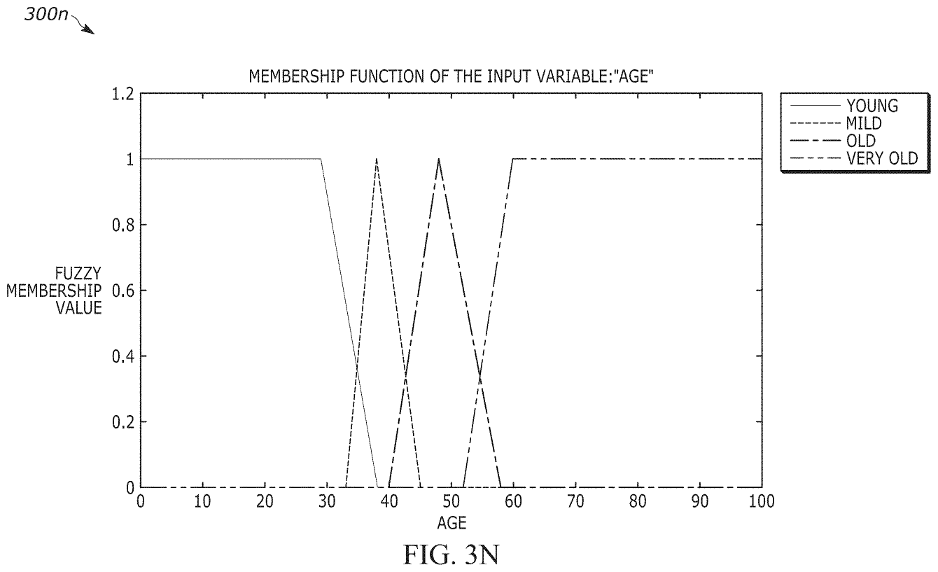

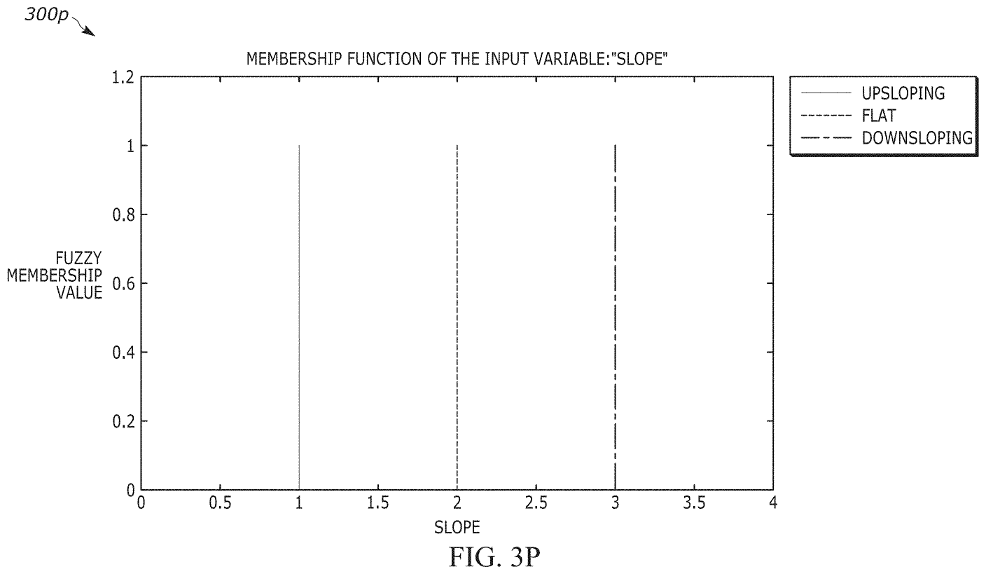

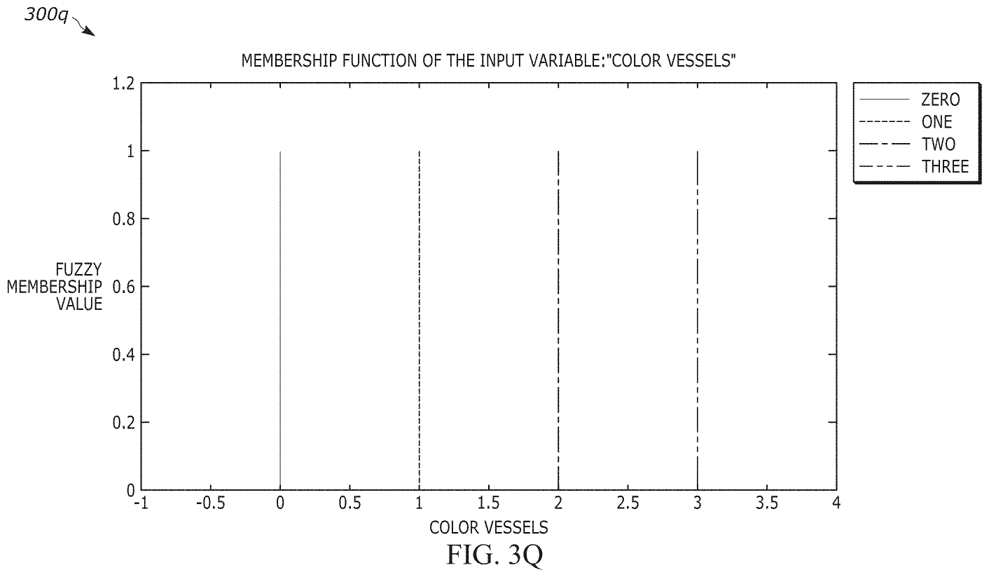

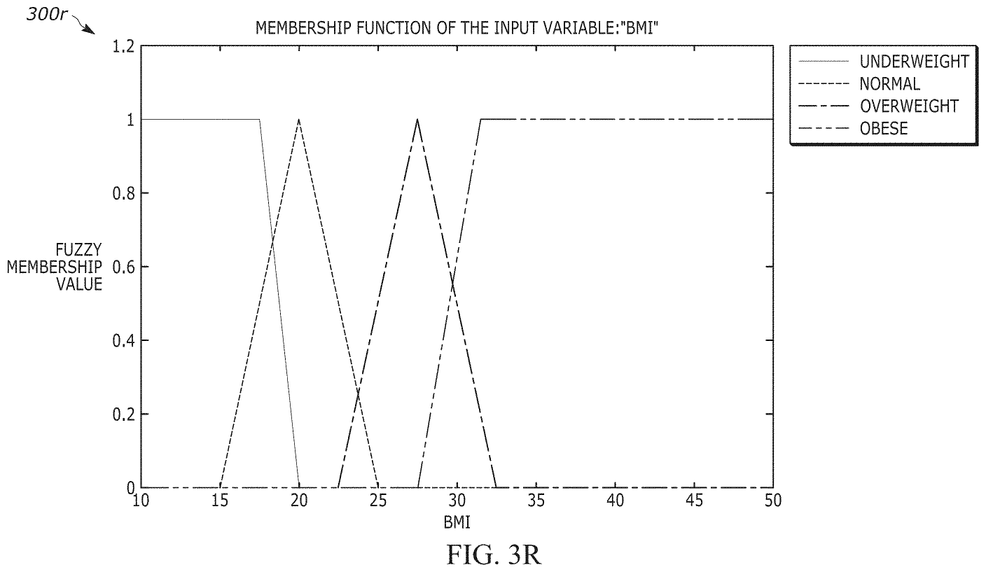

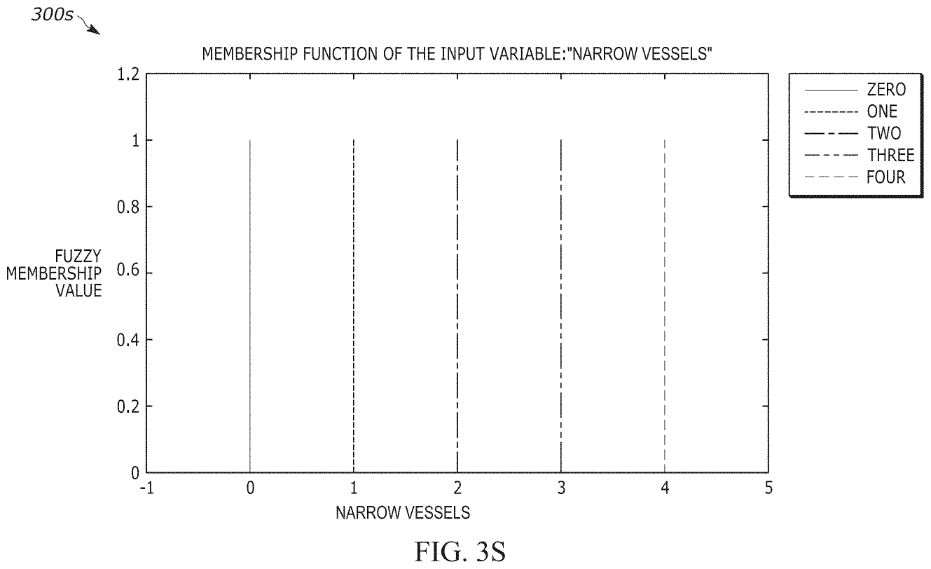

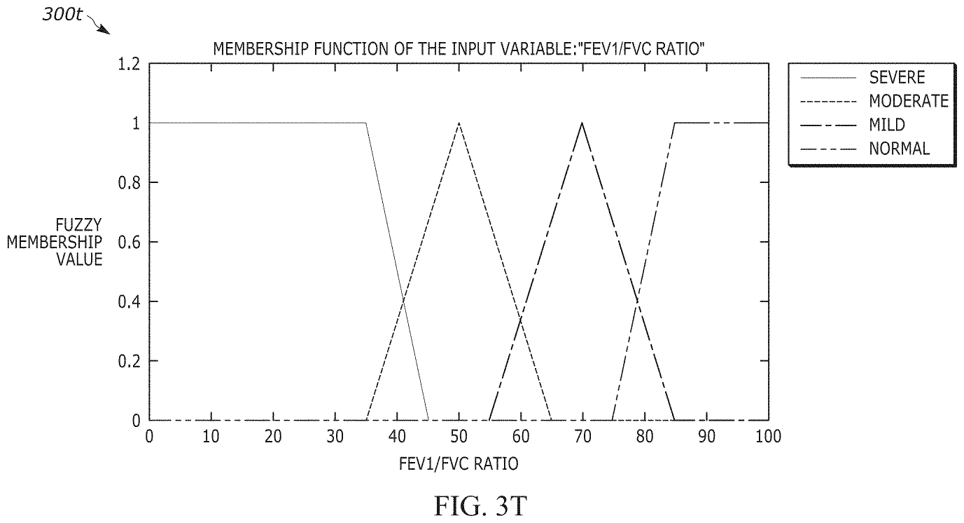

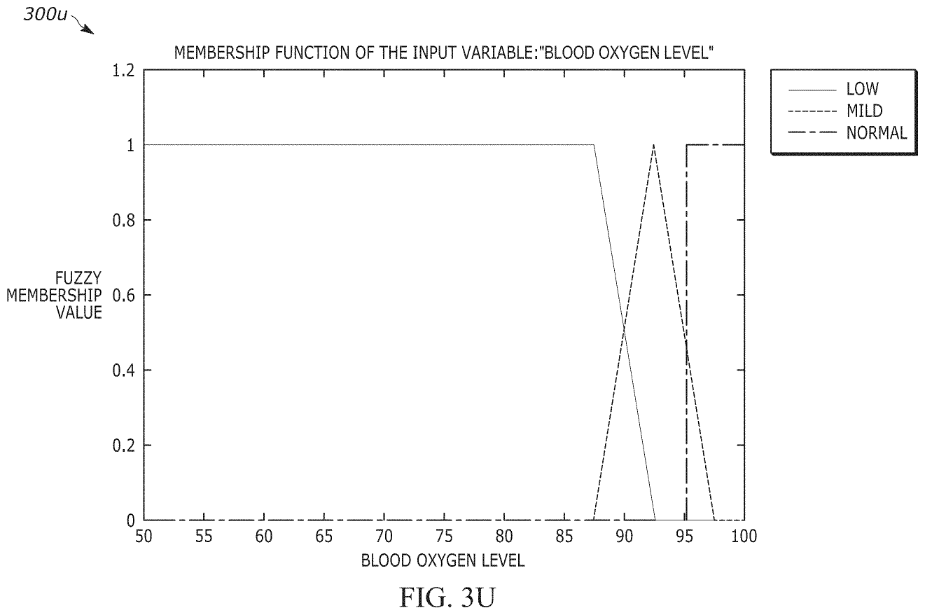

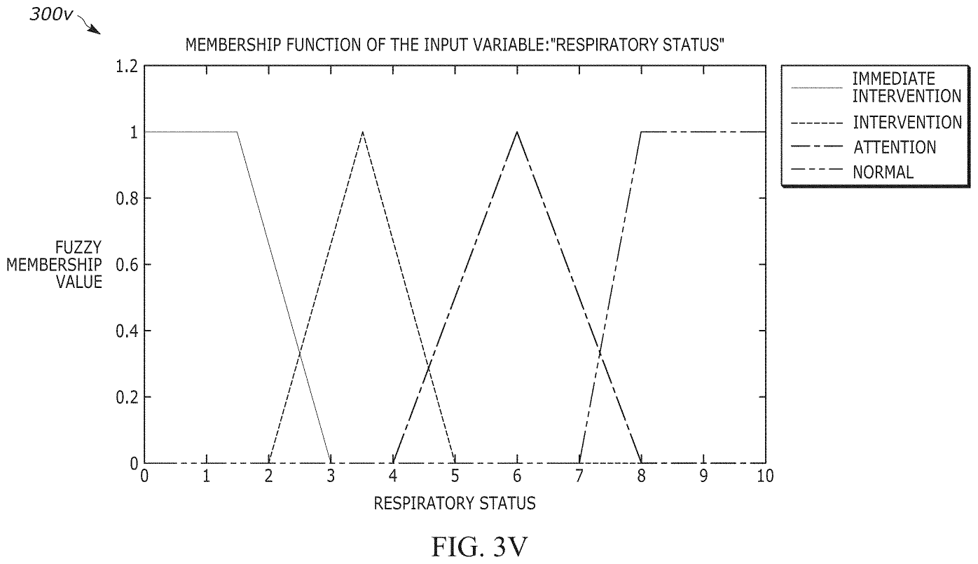

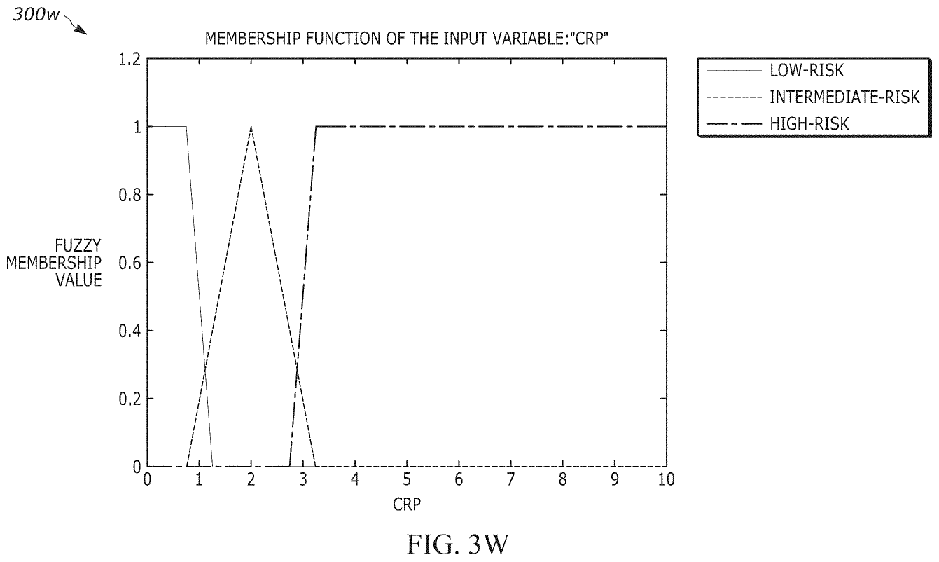

As one example, referring to FIG. 3A, the sub-variables 218 associated with chest pain may be converted into the fuzzy domain using one of Equations (1)-(4) below.

$${μ}_{{Typical}_{Angina}} ( x ) = \{ \begin{matrix}1 & x = 1 \\ 0 & otherwise\end{matrix} \qquad ( 1 )$$

$${μ}_{{Atypical}_{Angina}} ( x ) = \{ \begin{matrix}1 & x = 2 \\ 0 & otherwise\end{matrix} \qquad ( 2 )$$

$${μ}_{{Non}_{{Angina}_{Pain}}} ( x ) = \{ \begin{matrix}1 & x = 3 \\ 0 & otherwise\end{matrix} \qquad ( 3 )$$

$${μ}_{Asymptomatic} ( x ) = \{ \begin{matrix}1 & x = 4 \\ 0 & otherwise\end{matrix} \qquad ( 4 )$$

As another example, referring to FIG. 3B, the sub-variables 218 associated with blood pressure (systolic) may be converted into the fuzzy domain using one of Equations (5)-(8) below.

$${μ}_{{BP}_{{S}_{Low}}} ( x ) = \{ \begin{matrix}\begin{matrix}1 & x \leq 111\end{matrix} \\ \begin{matrix}\frac{134 - x}{23} & 111 < x \leq 134\end{matrix} \\ \begin{matrix}0 & x > 134\end{matrix}\end{matrix} \qquad ( 5 )$$

$${μ}_{{BP}_{{S}_{Medium}}} ( x ) = \{ \begin{matrix}\begin{matrix}0 & x\end{matrix} \leq 127 \\ \begin{matrix}\frac{x - 127}{12} & 127 < x \leq 139\end{matrix} \\ \begin{matrix}\frac{153 - x}{14} & 139 < x \leq 153\end{matrix} \\ \begin{matrix}0 & x > 153\end{matrix}\end{matrix} \qquad ( 6 )$$

$${μ}_{{BP}_{{S}_{High}}} ( x ) = \{ \begin{matrix}\begin{matrix}0 & x\end{matrix} \leq 142 \\ \begin{matrix}\frac{x - 142}{15} & 142 < x \leq 157\end{matrix} \\ \begin{matrix}\frac{172 - x}{15} & 157 < x \leq 172\end{matrix} \\ \begin{matrix}0 & x > 172\end{matrix}\end{matrix} \qquad ( 7 )$$

$${μ}_{{BP}_{{S}_{VeryHigh}}} ( x ) = \{ \begin{matrix}\begin{matrix}0 & x\end{matrix} \leq 154 \\ \begin{matrix}\frac{x - 154}{17} & 154 < x \leq 171\end{matrix} \\ \begin{matrix}1 & x > 171\end{matrix}\end{matrix} \qquad ( 8 )$$

As another example, referring to FIG. 3C, the sub-variables 218 associated with blood pressure (diastolic) may be converted into the fuzzy domain using one of Equations (9)-(13) below.

$${μ}_{{BP}_{{D}_{L}}} ( x ) = \{ \begin{matrix}\begin{matrix}1 & x \leq 60\end{matrix} \\ \begin{matrix}7 - \frac{x}{10} & 60 < x \leq 70\end{matrix} \\ \begin{matrix}0 & x > 70\end{matrix}\end{matrix} \qquad ( 9 )$$

$${μ}_{{BP}_{{D}_{N}}} ( x ) = \{ \begin{matrix}\begin{matrix}0 & x \leq 60\end{matrix} \\ \begin{matrix}\frac{x}{15} - 4 & 60 < x \leq 75\end{matrix} \\ \begin{matrix}6 - \frac{x}{15} & 75 < x \leq 90\end{matrix} \\ \begin{matrix}0 & x > 90\end{matrix}\end{matrix} \qquad ( 10 )$$

$${μ}_{{BP}_{{D}_{HS 1}}} ( x ) = \{ \begin{matrix}\begin{matrix}0 & x \leq 85\end{matrix} \\ \begin{matrix}\frac{x}{10} - 8.5 & 85 < x \leq 95\end{matrix} \\ \begin{matrix}10.5 - \frac{x}{10} & 95 < x \leq 105\end{matrix} \\ \begin{matrix}0 & x > 105\end{matrix}\end{matrix} \qquad ( 11 )$$

$${μ}_{{BP}_{{D}_{HS 2}}} ( x ) = \{ \begin{matrix}\begin{matrix}0 & x \leq 95\end{matrix} \\ \begin{matrix}\frac{x}{10} - 9.5 & 95 < x \leq 105\end{matrix} \\ \begin{matrix}11.5 - \frac{x}{10} & 105 < x \leq 115\end{matrix} \\ \begin{matrix}0 & x > 115\end{matrix}\end{matrix} \qquad ( 12 )$$

$${μ}_{{BP}_{{D}_{HC}}} ( x ) = \{ \begin{matrix}\begin{matrix}0 & x \leq 105\end{matrix} \\ \begin{matrix}\frac{x}{5} - 21 & 105 < x \leq 110\end{matrix} \\ \begin{matrix}1 & x > 110\end{matrix}\end{matrix} \qquad ( 13 )$$

As another example, referring to FIG. 3D, the sub-variables 218 associated with cholesterol may be converted into the fuzzy domain using one of Equations (14)-(17) below.

$${μ}_{{CL}_{Low}} ( x ) = \{ \begin{matrix}\begin{matrix}1 & x \leq 151\end{matrix} \\ \begin{matrix}\frac{197 - x}{46} & 151 < x \leq 197\end{matrix} \\ \begin{matrix}0 & x > 197\end{matrix}\end{matrix} \qquad ( 14 )$$

$${μ}_{{CL}_{Medium}} ( x ) = \{ \begin{matrix}\begin{matrix}0 & x \leq 188\end{matrix} \\ \begin{matrix}\frac{x - 188}{27} & 188 < x \leq 215\end{matrix} \\ \begin{matrix}\frac{250 - x}{35} & 215 < x \leq 250\end{matrix} \\ \begin{matrix}0 & x > 250\end{matrix}\end{matrix} \qquad ( 15 )$$

$${μ}_{{CL}_{High}} ( x ) = \{ \begin{matrix}\begin{matrix}0 & x \leq 217\end{matrix} \\ \begin{matrix}\frac{x - 217}{46} & 217 < x \leq 263\end{matrix} \\ \begin{matrix}\frac{307 - x}{44} & 263 < x \leq 307\end{matrix} \\ \begin{matrix}0 & x > 307\end{matrix}\end{matrix} \qquad ( 16 )$$

$${μ}_{{CL}_{VeryHigh}} ( x ) = \{ \begin{matrix}\begin{matrix}0 & x \leq 281\end{matrix} \\ \begin{matrix}\frac{x - 281}{66} & 281 < x \leq 347\end{matrix} \\ \begin{matrix}1 & x > 347\end{matrix}\end{matrix} \qquad ( 17 )$$

As another example, referring to FIG. 3E, the sub-variables 218 associated with blood sugar (diabetes) may be converted into the fuzzy domain using Equation (18) below.

$${μ}_{{BS}_{True}} ( x ) = \{ \begin{matrix}\begin{matrix}0 & x \leq 105\end{matrix} \\ \begin{matrix}\frac{x - 105}{15} & 105 < x \leq 120\end{matrix} \\ \begin{matrix}1 & x > 120\end{matrix}\end{matrix} \qquad ( 18 )$$

As another example, referring to FIG. 3F, the sub-variables 218 associated with resting electrocardiography may be converted into the fuzzy domain using one of Equations (19)-(21) below.

$${μ}_{{ECG}_{Normal}} ( x ) = \{ \begin{matrix}\begin{matrix}1 & x \leq 0.07\end{matrix} \\ \begin{matrix}\frac{0.4 - x}{0.33} & 0.07 < x \leq 0.4\end{matrix} \\ \begin{matrix}0 & x > 0.4\end{matrix}\end{matrix} \qquad ( 19 )$$

$${μ}_{{ECG}_{ST} - Abnormal} ( x ) = \{ \begin{matrix}\begin{matrix}0 & x \leq 0.25\end{matrix} \\ \begin{matrix}\frac{x - 0.25}{0.75} & 0.25 < x \leq 1\end{matrix} \\ \begin{matrix}\frac{1.75 - x}{0.75} & 1 < x \leq 1.75\end{matrix} \\ \begin{matrix}0 & x > 1.75\end{matrix}\end{matrix} \qquad ( 20 )$$

$${μ}_{Hypertrophy} ( x ) = \{ \begin{matrix}\begin{matrix}0 & x \leq 1.4\end{matrix} \\ \begin{matrix}\frac{x - 1.4}{0.4} & 1.4 < x \leq 1.8\end{matrix} \\ \begin{matrix}1 & x > 1.8\end{matrix}\end{matrix} \qquad ( 21 )$$

As another example, referring to FIG. 3G, the sub-variables 218 associated with maximum heart rate may be converted into the fuzzy domain using one of Equations (22)-(24) below.

$${μ}_{{MHR}_{Low}} ( x ) = \{ \begin{matrix}\begin{matrix}1 & x \leq 100\end{matrix} \\ \begin{matrix}\frac{141 - x}{41} & 100 < x \leq 141\end{matrix} \\ \begin{matrix}0 & x > 141\end{matrix}\end{matrix} \qquad ( 22 )$$

$${μ}_{{MHR}_{Medium}} ( x ) = \{ \begin{matrix}\begin{matrix}0 & x \leq 111\end{matrix} \\ \begin{matrix}\frac{x - 111}{41} & 111 < x \leq 152\end{matrix} \\ \begin{matrix}\frac{194 - x}{42} & 152 < x \leq 194\end{matrix} \\ \begin{matrix}0 & x > 194\end{matrix}\end{matrix} \qquad ( 23 )$$

$${μ}_{{MHR}_{High}} ( x ) = \{ \begin{matrix}\begin{matrix}0 & x \leq 152\end{matrix} \\ \begin{matrix}\frac{x - 152}{64} & 152 < x \leq 216\end{matrix} \\ \begin{matrix}1 & x > 216\end{matrix}\end{matrix} \qquad ( 24 )$$

As another example, referring to FIG. 3H, the sub-variables 218 associated with exercise may be converted into the fuzzy domain using Equation (25) below.

$${μ}_{ETrue} ( x ) = \{ \begin{matrix}1 & x = 1 \\ 0 & otherwise\end{matrix} \qquad ( 25 )$$

As another example, referring to FIG. 3J, the sub-variables 218 associated with old peak may be converted into the fuzzy domain using one of Equations (26)-(28) below.

$${μ}_{{OP}_{Low}} ( x ) = \{ \begin{matrix}\begin{matrix}1 & x \leq 1\end{matrix} \\ \begin{matrix}2 - x & 1 < x \leq 2\end{matrix} \\ \begin{matrix}0 & x > 2\end{matrix}\end{matrix} \qquad ( 26 )$$

$${μ}_{{OP}_{Risk}} ( x ) = \{ \begin{matrix}\begin{matrix}0 & x \leq 1.5\end{matrix} \\ \begin{matrix}\frac{x - 1.5}{1.3} & 1.5 < x \leq 2.8\end{matrix} \\ \begin{matrix}\frac{4.2 - x}{1.4} & 2.8 < x \leq 4.2\end{matrix} \\ \begin{matrix}0 & x > 4.2\end{matrix}\end{matrix} \qquad ( 27 )$$

$${μ}_{{OP}_{Terrible}} ( x ) = \{ \begin{matrix}\begin{matrix}0 & x \leq 2.55\end{matrix} \\ \begin{matrix}\frac{x - 2.55}{1.45} & 2.55 < x \leq 4\end{matrix} \\ \begin{matrix}1 & x > 4\end{matrix}\end{matrix} \qquad ( 28 )$$

As another example, referring to FIG. 3K, the sub-variables 218 associated with thallium scan may be converted into the fuzzy domain using one of Equations (29)-(31) below.

$${μ}_{{TS}_{Normal}} ( x ) = \{ \begin{matrix}1 & x = 3 \\ 0 & otherwise\end{matrix} \qquad ( 29 )$$

$${μ}_{{TS}_{FixedDefect}} ( x ) = \{ \begin{matrix}1 & x = 6 \\ 0 & otherwise\end{matrix} \qquad ( 30 )$$

$${μ}_{{TS}_{Reversible}} ( x ) = \{ \begin{matrix}0 & x = 7 \\ 1 & otherwise\end{matrix} \qquad ( 31 )$$

As another example, referring to FIG. 3M, the sub-variables 218 associated with sex may be converted into the fuzzy domain using one of Equations (32)-(33) below.

$${μ}_{{Sex}_{Male}} ( x ) = \{ \begin{matrix}1 & x = 1 \\ 0 & x = 0\end{matrix} \qquad ( 32 )$$

$${μ}_{{Sex}_{Female}} ( x ) = \{ \begin{matrix}0 & x = 1 \\ 1 & x = 0\end{matrix} \qquad ( 33 )$$

As another example, referring to FIG. 3N, the sub-variables 218 associated with age may be converted into the fuzzy domain using one of Equations (34)-(37) below.

$${μ}_{{Age}_{Young}} ( x ) = \{ \begin{matrix}1 & x \leq 29 \\ \frac{38 - x}{9} & 29 < x \leq 38 \\ 0 & x > 38\end{matrix} \qquad ( 34 )$$

$${μ}_{{Age}_{Mild}} ( x ) = \{ \begin{matrix}0 & x \leq 33 \\ \frac{x - 33}{5} & 33 < x \leq 38 \\ \frac{45 - x}{7} & 38 < x \leq 45 \\ 0 & x > 45\end{matrix} \qquad ( 35 )$$

$${μ}_{{Age}_{Old}} ( x ) = \{ \begin{matrix}0 & x \leq 40 \\ \frac{x - 40}{8} & 40 < x \leq 48 \\ \frac{58 - x}{10} & 48 < x \leq 58 \\ 0 & x > 58\end{matrix} \qquad ( 36 )$$

$${μ}_{{Age}_{VeryOld}} ( x ) = \{ \begin{matrix}0 & x \leq 2. 55 \\ \frac{x - 2.55}{1.45} & 2.55 < x \leq 4 \\ 1 & x > 4\end{matrix} \qquad ( 37 )$$

As another example, referring to FIG. 3P, the sub-variables 218 associated with slope may be converted into the fuzzy domain using one of Equations (38)-(40) below.

$${μ}_{{Slope}_{Up}} ( x ) = \{ \begin{matrix}1 & x = 1 \\ 0 & otherwise\end{matrix} \qquad ( 38 )$$

$${μ}_{{Slope}_{Flat}} ( x ) = \{ \begin{matrix}1 & x = 2 \\ 0 & otherwise\end{matrix} \qquad ( 39 )$$

$${μ}_{{Slope}_{Down}} ( x ) = \{ \begin{matrix}1 & x = 3 \\ 0 & otherwise\end{matrix} \qquad ( 40 )$$

As another example, referring to FIG. 3Q, the sub-variables 218 associated with color vessels may be converted into the fuzzy domain using one of Equations (41)-(44) below.

$${μ}_{Zero} ( x ) = \{ \begin{matrix}1 & x = 0 \\ 0 & otherwise\end{matrix} \qquad ( 41 )$$

$${μ}_{One} ( x ) = \{ \begin{matrix}1 & x = 1 \\ 0 & otherwise\end{matrix} \qquad ( 42 )$$

$${μ}_{Two} ( x ) = \{ \begin{matrix}1 & x = 2 \\ 0 & otherwise\end{matrix} \qquad ( 43 )$$

$${μ}_{Three} ( x ) = \{ \begin{matrix}1 & x = 3 \\ 0 & otherwise\end{matrix} \qquad ( 44 )$$

As another example, referring to FIG. 3R, the sub-variables 218 associated with BMI may be converted into the fuzzy domain using one of Equations (45)-(48) below.

$${μ}_{{BMI}_{UW}} ( x ) = \{ \begin{matrix}1 & x \leq 17. 5 \\ 8 - 0.4 x & 17.5 < x \leq 20 \\ 0 & x > 20\end{matrix} \qquad ( 45 )$$

$${μ}_{{BMI}_{Normal}} ( x ) = \{ \begin{matrix}0 & x \leq 15 \\ 0.2 x - 3 & 15 < x \leq 20 \\ 5 - 0.2 x & 20 < x \leq 25 \\ 0 & x > 25\end{matrix} \qquad ( 46 )$$

$${μ}_{{BMI}_{OW}} ( x ) = \{ \begin{matrix}0 & x \leq 22.5 \\ 0.2 x - 4.5 & 22.5 < x \leq 27.5 \\ 6.5 - 0 \text{.2} x & 27.5 < x \leq 32.5 \\ 0 & x > 32.5\end{matrix} \qquad ( 47 )$$

$${μ}_{{BMI}_{Obese}} ( x ) = \{ \begin{matrix}0 & x \leq 27. 5 \\ 0.25 x - 6 \text{.875} & 27.5 < x \leq 31.5 \\ 1 & x > 31. 5\end{matrix} \qquad ( 48 )$$

As another example, referring to FIG. 3S, the sub-variables 218 associated with narrow vessels may be converted into the fuzzy domain using one of Equations (49)-(53) below.

$${μ}_{Zero} ( x ) = \{ \begin{matrix}1 & x = 0 \\ 0 & otherwise\end{matrix} \qquad ( 49 )$$

$${μ}_{One} ( x ) = \{ \begin{matrix}1 & x = 1 \\ 0 & otherwise\end{matrix} \qquad ( 50 )$$

$${μ}_{T w o} ( x ) = \{ \begin{matrix}1 & x = 2 \\ 0 & otherwise\end{matrix} \qquad ( 51 )$$

$${μ}_{Three} ( x ) = \{ \begin{matrix}1 & x = 3 \\ 0 & otherwise\end{matrix} \qquad ( 52 )$$

$${μ}_{Four} ( x ) = \{ \begin{matrix}1 & x = 4 \\ 0 & otherwise\end{matrix} \qquad ( 53 )$$

As another example, referring to FIG. 3T, the sub-variables 218 associated with FEV1/FVC ratio may be converted into the fuzzy domain using one of Equations (54)-(57) below.

$${μ}_{{FFR}_{Severe}} ( x ) = \{ \begin{matrix}1 & 0 \leq x \leq 35 \\ \frac{9}{2} - \frac{x}{10} & 35 < x \leq 45 \\ 0 & x > 45\end{matrix} \qquad ( 54 )$$

$${μ}_{{FFR}_{Moderate}} ( x ) = \{ \begin{matrix}0 & x \leq 35 \\ \frac{x}{15} - \frac{7}{3} & 35 < x \leq 50 \\ \frac{13}{3} - \frac{x}{15} & 50 < x \leq 65 \\ 0 & x > 65\end{matrix} \qquad ( 55 )$$

$${μ}_{{FFR}_{Mild}} ( x ) = \{ \begin{matrix}0 & x \leq 55 \\ \frac{x}{15} - \frac{11}{3} & 55 < x \leq 70 \\ \frac{17}{3} - \frac{x}{15} & 70 < x \leq 85 \\ 0 & x > 85\end{matrix} \qquad ( 56 )$$

$${μ}_{{FFR}_{Normal}} ( x ) = \{ \begin{matrix}0 & 0 \leq x \leq 75 \\ \frac{x}{10} - \frac{15}{2} & 75 < x \leq 85 \\ 1 & x > 85\end{matrix} \qquad ( 57 )$$

As another example, referring to FIG. 3U, the sub-variables 218 associated with blood oxygen level may be converted into the fuzzy domain using one of Equations (58)-(60) below.

$${μ}_{{BOL}_{Low}} ( x ) = \{ \begin{matrix}1 & 0 \leq x \leq 87.5 \\ 18.5 - \frac{x}{5} & 87.5 < x \leq 92.5 \\ 0 & x > 92.5\end{matrix} \qquad ( 58 )$$

$${μ}_{{BOL}_{Mild}} ( x ) = \{ \begin{matrix}0 & x \leq 87. 5 \\ \frac{x}{5} - 17.5 & 87.5 < x \leq 92.5 \\ 19.5 - \frac{x}{5} & 92.5 < x \leq 97.5 \\ 0 & x > 97. 5\end{matrix} \qquad ( 59 )$$

$${μ}_{{BOL}_{Normal}} ( x ) = \{ \begin{matrix}0 & 0 \leq x \leq 92.5 \\ \frac{x}{2.5} - 45 & 92.5 < x \leq 95 \\ 1 & x > 95\end{matrix} \qquad ( 60 )$$

As another example, referring to FIG. 3V, the sub-variables 218 associated with respiratory status may be converted into the fuzzy domain using one of Equations (61)-(64) below.

$${μ}_{R {S}_{Normal}} ( x ) = \{ \begin{matrix}0 & 0 \leq x \leq 7 \\ x - 7 & 7 < x \leq 8 \\ 1 & x > 8\end{matrix} \qquad ( 61 )$$

$${μ}_{R {S}_{Attention}} ( x ) = \{ \begin{matrix}0 & x \leq 4 \\ \frac{x}{2} - 2 & 4 < x \leq 6 \\ 4 - \frac{x}{2} & 6 < x \leq 8 \\ 0 & x > 8\end{matrix} \qquad ( 62 )$$

$${μ}_{R {S}_{Intervention}} ( x ) = \{ \begin{matrix}0 & x \leq 2 \\ \frac{x}{1.5} - \frac{4}{3} & 2 < x \leq 3.5 \\ \frac{10}{3} - \frac{x}{1.5} & 3.5 < x \leq 5 \\ 0 & x > 5\end{matrix} \qquad ( 63 )$$

$${μ}_{R {S}_{ImmediateIntervention}} ( x ) = \{ \begin{matrix}1 & 0 \leq x \leq 1.5 \\ 2 - \frac{x}{1.5} & 1.5 < x \leq 3 \\ 0 & x > 3\end{matrix} \qquad ( 64 )$$

As another example, referring to FIG. 3W, the sub-variables 218 associated with CRP may be converted into the fuzzy domain using one of Equations (65)-(67) below.

$${μ}_{{CRP}_{Low} - R i s k} ( x ) = \{ \begin{matrix}1 & 0 \leq x \leq 0.75 \\ 2.5 - 2 x & 0.75 < x \leq 1.25 \\ 0 & x > 1.2 5\end{matrix} \qquad ( 65 )$$

$${μ}_{{CRP}_{Intermediate} - R i s k} ( x ) = \{ \begin{matrix}0 & x \leq 0.7 5 \\ 0.8 x - 0.6 & 0.75 < x \leq 2 \\ 2.6 - 0.8 x & 2 < x \leq 3. 25 \\ 0 & x > 3.2 5\end{matrix} \qquad ( 66 )$$

$${μ}_{{CRP}_{High} - R i s k} ( x ) = \{ \begin{matrix}0 & 0 \leq x \leq 2.75 \\ 2.5 x - 6 \text{.875} & 2.75 < x \leq 3. 15 \\ 1 & x > 3.1 5\end{matrix} \qquad ( 67 )$$

Referring to FIG. 2, the fuzzy domain module 206 is configured to transmit the fuzzy sub-variables 218 to the fuzzy inference module 208. The fuzzy inference module 208 receives a plurality of rules 500 from a fuzzy rule base module 210 to formulate the mapping from the fuzzy sub-variables 218 to output variables 220 (e.g., “Healthy,” “Sick1,” “Sick2,” “Sick3,” and “Sick4”) using fuzzy logic. The fuzzy inference module 208 is configured to apply reasoning to compute the fuzzy output variables 220.

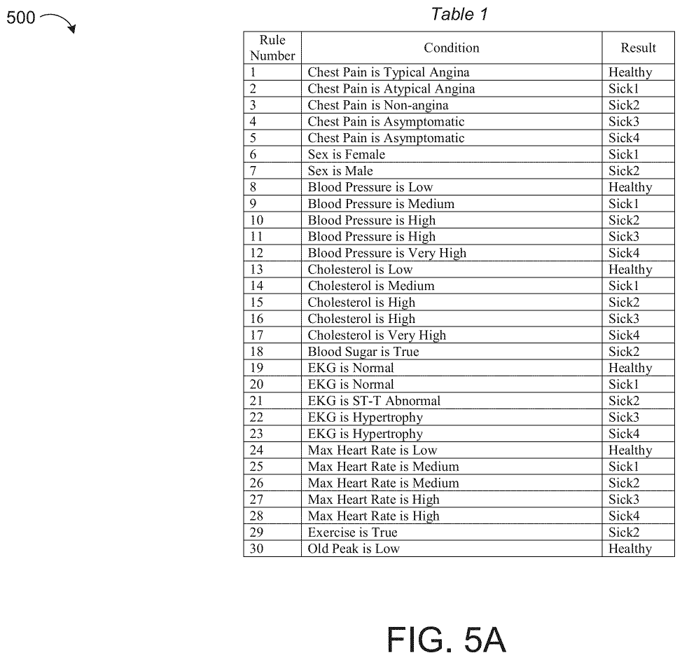

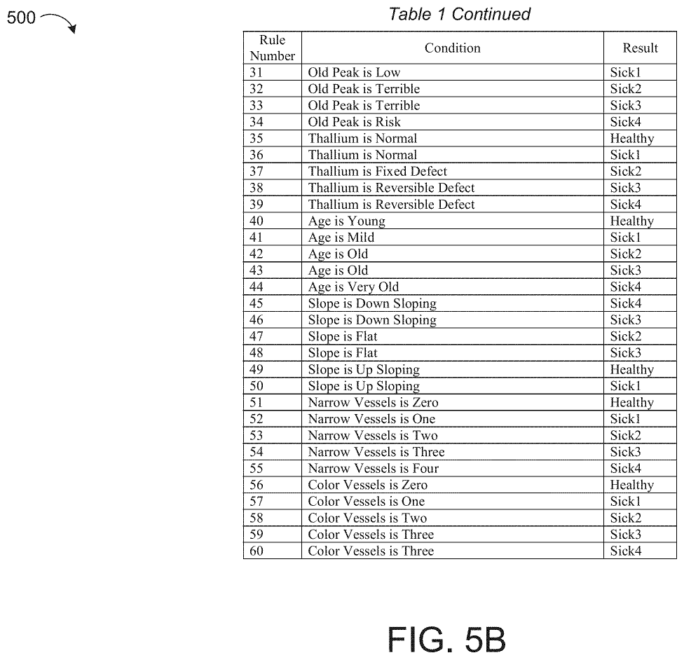

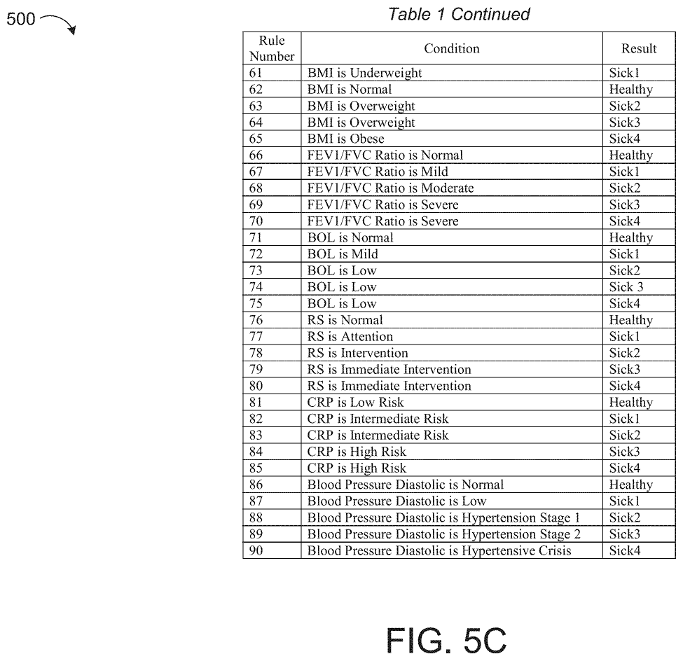

The plurality of rules 500 from the fuzzy rule base module 210 may include ninety (90) rules as shown in Table 1 in FIGS. 5A-5C, with twenty input variables (e.g., the fuzzy sub-variables 218) and five output variables (e.g., the fuzzy output variables 220). In other implementations, the fuzzy rule base module 210 may include any suitable number of rules, input variables, and output variables.

As one example, referring to FIG. 5B and the rules associated with the number of narrow vessels (i.e., rule numbers 51-55), if the number of narrow vessels is equal to zero, the fuzzy inference module 208 determines, based on the applicable rule from the fuzzy rule base module 210, that the result is “Healthy,” if the number of narrow vessels is equal to one, the fuzzy inference module 208 determines, based on the applicable rule from the fuzzy rule base module 210, that the result is “Sick1,” if the number of narrow vessels is equal to two, the fuzzy inference module 208 determines, based on the applicable rule from the fuzzy rule base module 210, that the result is “Sick2,” if the number of narrow vessels is equal to three, the fuzzy inference module 208 determines, based on the applicable rule from the fuzzy rule base module 210, that the result is “Sick3,” and if the number of narrow vessels is equal to four, the fuzzy inference module 208 determines, based on the applicable rule from the fuzzy rule base module 210, that the result is “Sick4.” The results determined by the fuzzy inference module 208 indicate a degree of health on a scale, with “Healthy” being the most healthy and “Sick4” being the most sick.

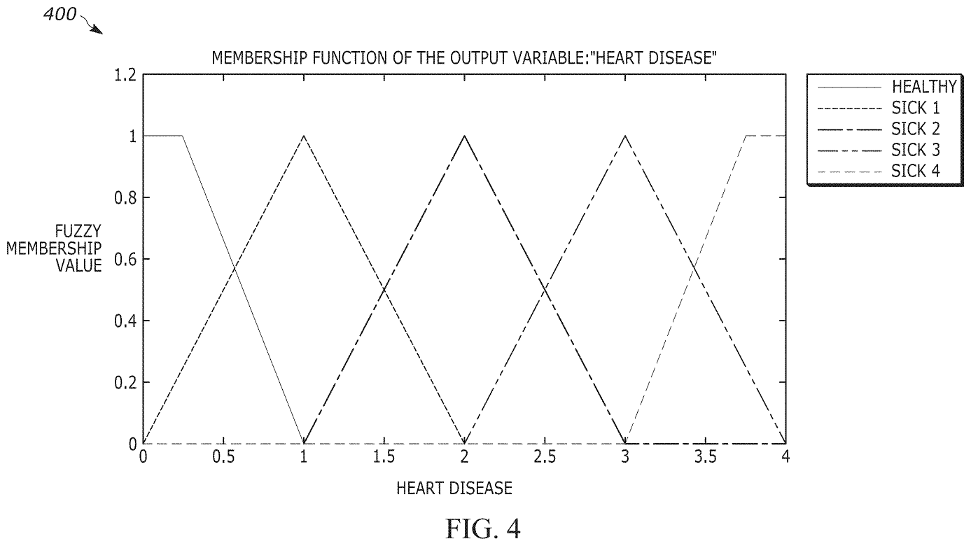

Referring to FIG. 4, the fuzzy inference module 208 is configured to compute fuzzy outputs for the output variables 220 using Equations (68)-(72) below.

$${μ}_{{HD}_{Healthy}} ( x ) = \{ \begin{matrix}1 & x \leq 0.2 5 \\ \frac{1 - x}{0.75} & 0.25 < x \leq 1 \\ 0 & x > 1\end{matrix} \qquad ( 68 )$$

$${μ}_{{HD}_{Sick 1}} ( x ) = \{ \begin{matrix}x & x \leq 1 \\ 2 - x & 1 < x \leq 2 \\ 0 & x > 2\end{matrix} \qquad ( 69 )$$

$${μ}_{{HD}_{Sick 2}} ( x ) = \{ \begin{matrix}0 & x \leq 1 \\ x - 1 & 1 < x \leq 2 \\ 3 - x & 2 < x \leq 3 \\ 0 & x > 3\end{matrix} \qquad ( 70 )$$

$${μ}_{{HD}_{Sick 3}} ( x ) = \{ \begin{matrix}0 & x \leq 2 \\ x - 2 & 2 < x \leq 3 \\ 4 - x & 3 < x \leq 4 \\ 0 & x > 4\end{matrix} \qquad ( 71 )$$

$${μ}_{{HD}_{Sick 4}} ( x ) = \{ \begin{matrix}0 & x \leq 3 \\ \frac{x - 3}{0.75} & 3 < x \leq 3.75 \\ 1 & x > 3.7 5\end{matrix} \qquad ( 72 )$$

Referring to FIG. 2, the fuzzy inference module 208 is configured to transmit the fuzzy sub-variables 218 and the fuzzy output variables 220 to the deffuzification module 212. The deffuzification module 212 is configured to produce a quantifiable result in crisp logic, based on the fuzzy sub-variables 218 and the corresponding fuzzy output variables 220 (e.g., the corresponding membership degrees). The deffuzification module 212 is configured to map a fuzzy set to a crisp set. In some implementations, the deffuzification module 212 may use one or more of the following techniques to map a fuzzy set to a crisp set: Weighted Average Formulate (WAF) Method, Quality Method (QM), Maximum-Weighted Average Formula (MAX-WAF) Method, and Center of Sums (COS) Method. In some implementations, the deffuzification module 212 may determine the average of the results of the four above techniques to determine a heart disease diagnosis 222 for the patient 101. In some implementations, the heart disease diagnosis 222 may be a numerical value associated with one of the output variables 220 (e.g., “Healthy,” “Sick1,” “Sick2,” “Sick3,” and “Sick4”).

A collection of N inference rules for a system with k input variables and one output variable and whose form is typical in fuzzy decision system is such that the j^{th} rule is defined as the following Equation (73):

Rⱼ:If x is Aⱼ, then y is Bⱼ

Where 1≤j≤N

x represents input variables

x∈[x₁,x₂, . . . ,xᵢ, . . . ,xₖ]

y represents output variables (73)

If Aⱼ(xⱼ)=uⱼ, then with Bⱼ denoting the crisp output of rule j, the deffuzification module 212 may apply the WAF method as follows in Equation (74) to produce the crisp output C:

$${C}_{WAF} = \frac{{\sum }_{j = 1}^{N} {B}_{j} {u}_{j}}{{\sum }_{j = 1}^{N} {u}_{j}} \qquad ( 74 )$$

When more than one fuzzy rule possesses the same crisp consequent, then an “or” operator between such conflicted rules may be reflected through the use of the maximum operation applied to the membership grades resulting from these rules. The output variable may be divided into p sub-variables. Then, O₁, O₂, . . . , Oⱼ, . . . , Oₚ may be equal to sets of membership grades that belong to 1^{st}, 2^{nd}, . . . , j^{th}, . . . , p^{th} sub-variables, respectively. The deffuzification module 212 may apply the MAX-WAF method, wherein the crisp output C is calculated per Equation (75) below:

$${C}_{M A X - W A F} = \frac{{\sum }_{j = 1}^{p} {B}_{j} {u}_{j}}{{\sum }_{j = 1}^{p} {u}_{j}} \qquad ( 75 )$$

If Aⱼ(xᵢ)=uⱼ, then with Bⱼ denoting the crisp output of rule j dⱼ being the measure of the support of the consequent of rule j, the deffuzification module 212 may apply the QM as follows in Equation (76) to produce the crisp output C:

$${C}_{Q M} = \frac{{\sum }_{j = 1}^{N} \frac{{B}_{j} {u}_{j}}{{d}_{j}}}{{\sum }_{j = 1}^{p} \frac{{u}_{j}}{{d}_{j}}} \qquad ( 76 )$$

If output fuzzy set B=B₁∪B₂, . . . , Bⱼ, . . . , ∪Bₚ, then the deffuzification module 212 may apply the COS as follows in Equation (77) to produce the crisp output C:

$${C}_{COS} = \frac{{\sum }_{j = 1}^{p} {x}_{j} {Area}_{{B}_{j}}}{{\sum }_{j = 1}^{p} {Area}_{{B}_{j}}} \qquad ( 77 )$$

Where Area_{B}_{ⱼ} denotes the area of the region bounded by fuzzy sets Bⱼ and xⱼ is the geometric center of the area Area_{B}_{ⱼ}.

Referring to FIG. 2, the deffuzification module 212 may transmit the heart disease diagnosis 222 and the corresponding output variable 220 to the output module 214. The output module 214 is configured to output and display the heart disease diagnosis 222 and the corresponding output variable 220. Additionally, in circumstances where the heart disease diagnosis 222 indicates that the patient 101 has a risk of developing heart disease, the output module 214 may output a treatment method to treat heart disease, prevent heart disease from developing, and/or treat symptoms associated with heart disease. For example, the output module 214 may transmit data indicating a dosage amount to the administration computing device 103b of the administration device 103. The administration computing device 103b may instruct the doser 103a to administer the dosage amount to the patient 101.

Referring to FIG. 5, the heart disease diagnosis GUI 600 is generally shown. The heart disease diagnosis GUI 600 may be executed by the HCP system 130 (e.g., the data processing hardware 132 and/or the computing resources 144) and displayed on the display 136 to be viewable and interacted with by the HCP 108. The heart disease diagnosis GUI 600 illustrates a plurality of input GUIs 602 and an output GUI 604.

The plurality of input GUIs 602 may include: a sex GUI 606 associated with the input data 216 and the input sub-variable 218 corresponding to sex; a chest pain GUI 608 associated with the input data 216 and the input sub-variable 218 corresponding to chest pain; a blood pressure GUI 610 associated with the input data 216 and the input sub-variable 218 corresponding to blood pressure (in some implementations, the blood pressure GUI 610 may include two GUIs, one associated with blood pressure systolic and the other associated with blood pressure diastolic); a cholesterol GUI 612 associated with the input data 216 and the input sub-variable 218 corresponding to cholesterol; a blood sugar GUI 614 associated with the input data 216 and the input sub-variable 218 corresponding to blood sugar; an ECG GUI 616 associated with the input data 216 and the input sub-variable 218 corresponding to ECG; a max heart rate GUI 618 associated with the input data 216 and the input sub-variable 218 corresponding to max heart rate; an exercise GUI 620 associated with the input data 216 and the input sub-variable 218 corresponding to exercise; an old peak GUI 622 associated with the input data 216 and the input sub-variable 218 corresponding to old peak; a thallium scan GUI 624 associated with the input data 216 and the input sub-variable 218 corresponding to thallium scan; an age GUI 626 associated with the input data 216 and the input sub-variable 218 corresponding to age; a slope GUI 628 associated with the input data 216 and the input sub-variable 218 corresponding to slope; a color vessels GUI 630 associated with the input data 216 and the input sub-variable 218 corresponding to color vessels; a BMI GUI 632 associated with the input data 216 and the input sub-variable 218 corresponding to BMI; a narrow vessels GUI 634 associated with the input data 216 and the input sub-variable 218 corresponding to narrow vessels; a FEV1/FEV ratio GUI 636 associated with the input data 216 and the input sub-variable 218 corresponding to FEV1/FEV ratio; a blood-oxygen level GUI 638 associated with the input data 216 and the input sub-variable 218 corresponding to blood-oxygen level; a respiratory status GUI 640 associated with the input data 216 and the input sub-variable 218 corresponding to blood-oxygen level; and a CRP GUI 642 associated with the input data 216 and the input sub-variable 218 corresponding to CRP.

The output GUI 604 includes a heart disease quantification GUI 644 and a heart disease scale GUI 646. The heart disease quantification GUI 644 displays a numerical value associated with the heart disease diagnosis 222. For example, as shown in FIG. 6, the heart disease quantification GUI 644 may display the value “0.9625,” which is the result determined by the heart disease diagnosis application 200 based on the input data 216 associated with that particular patient. The heart disease scale GUI 646 displays a scale from 0 to 4 and a line representing the numerical value displayed in the heart disease quantification GUI 644. The scale from 0 to 4 represents the output variables 220. For example, 0 to 1 equals “Healthy,” 1 to 2 equals “Sick1,” 2 to 3 equals “Sick2,” 3 to 4 equals “Sick3,” and greater than 4 equals “Sick4.” The heart disease scale GUI 646 may include a color gradient or any other suitable graphical display elements.

The input GUIs 602 (i.e., the input data 216 and input sub-variables 218 associated with and displayed by the input GUIs 602) may be manipulated and interacted with by the HCP 108. In some implementations, the diagnosis application 200 (e.g., the data processing hardware 132 and/or the computing resources 144) obtains (e.g., fetches or receives) the input data 216 from the patient device 102, the data collection device(s) 104, the patient records 110 and/or the external data 112. In other implementations, the HCP 108 may manually enter the input data 216, or some combination of the HCP 108 manually entering the input data 216 and the diagnosis application 200 obtaining the input data 216.

The heart disease diagnosis system 100 described herein may provide an alternative to the subjective opinions of healthcare providers related to heart disease diagnosis to avoid anomalies that may be the result of subjectiveness, human error, and/or slow process. The system 100 may benefit patients by providing early detection of abnormalities in heart conditions, thus, allowing these conditions to be treated at an early stage.

The heart disease diagnosis system 100 may determine the heart disease diagnosis 222 and the corresponding output variable 220 by implementing any suitable artificial intelligence system. For example, the system 100 may implement supervised or unsupervised artificial intelligence to determine the heart disease diagnosis 222. That is, in some implementations, the HCP 108 may supervise and/or train the artificial intelligence system, and in other implementations, the system 100 may operate with no supervision.

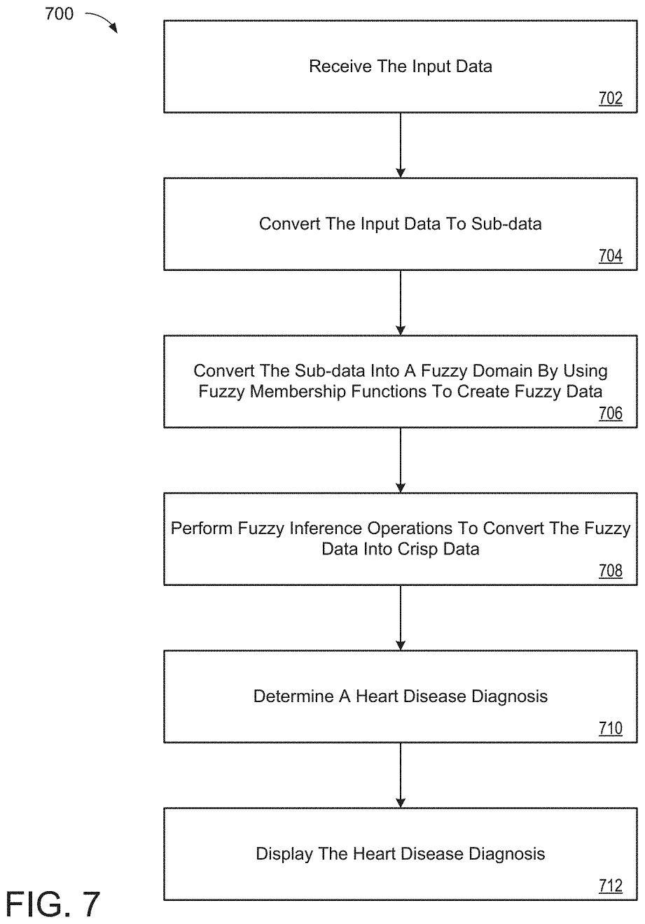

Referring to FIG. 7, a method 700 for determining a heart disease diagnosis using the heart disease diagnosis system 100 is generally shown. At step 702, the diagnosis application 200 (e.g., the data processing hardware 132 and/or the computing resources 144) may receive the input data 216 from the patient device 102, the data collection device(s) 104, the patient records 110 and/or the external data 112. At step 704, the diagnosis application 200 may convert the input data 216 to the sub-data or sub-variables 218. At step 706, the diagnosis application 200 may convert the sub-variables 218 into the fuzzy domain by using fuzzy membership functions to create fuzzy data. At step 708, the diagnosis application 200 may perform fuzzy inference operations to convert the fuzzy data into crisp data. At step 710, the diagnosis application 200 may determine and display the heart disease diagnosis 222 and the corresponding output variable 220. At step 712, the diagnosis application 200 may output a dose of medication to the administration device 103 to be administered to the patient 101. The foregoing steps of method 700 may include additional steps, fewer steps, or alternative steps as suitable.

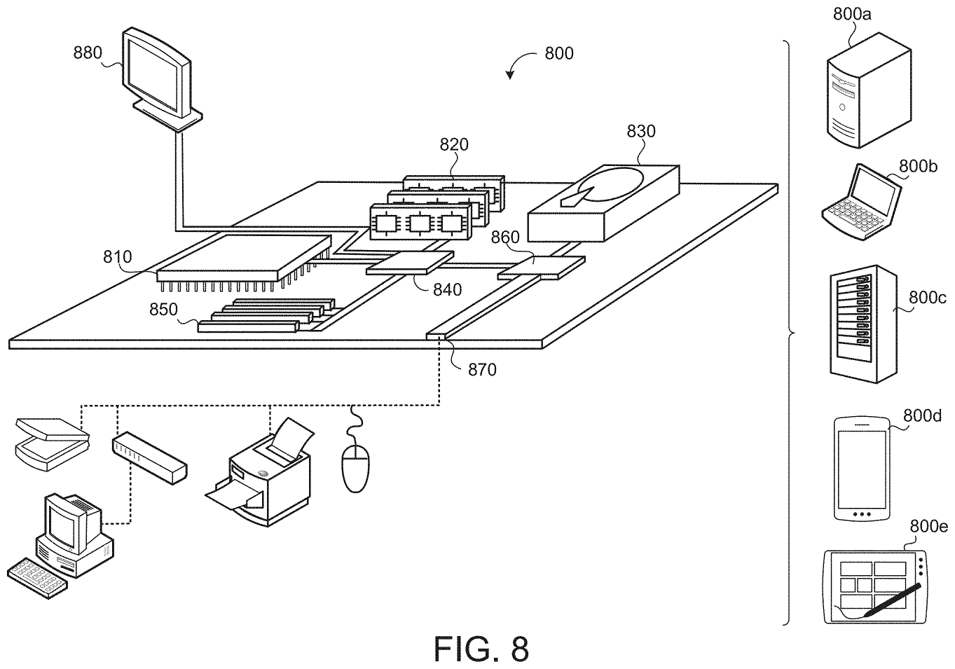

FIG. 8 is a schematic view of an example electronic device 800 (e.g., a computing device) that may be used to implement the systems and methods described in this document. The electronic device 800 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The electronic device 800 includes a processor 810, memory 820, a storage device 830, a high-speed interface/controller 840 connecting to the memory 820 and high-speed expansion ports 850, and a low speed interface/controller 860 connecting to a low speed bus 870 and a storage device 830. Each of the components 810, 820, 830, 840, 850, and 860, is interconnected using various busses, and may be mounted on a common motherboard or in other manners as appropriate. The processor 810 can process instructions for execution within the electronic device 800, including instructions stored in the memory 820 or on the storage device 830 to display graphical information for a graphical user interface (GUI) on an external input/output device, such as display 880 coupled to high speed interface 840. In other implementations, multiple processors and/or multiple buses may be used, as appropriate, along with multiple memories and types of memory. Also, multiple electronic device 800 may be connected, with each device providing portions of the necessary operations (e.g., as a server bank, a group of blade servers, or a multi-processor system).

The memory 820 stores information non-transitorily within the electronic device 800. The memory 820 may be a computer-readable medium, a volatile memory unit(s), or non-volatile memory unit(s). The non-transitory memory 820 may be physical devices used to store programs (e.g., sequences of instructions) or data (e.g., program state information) on a temporary or permanent basis for use by the electronic device 800. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 830 is capable of providing mass storage for the electronic device 800. In some implementations, the storage device 830 is a computer-readable medium. In various different implementations, the storage device 830 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 820, the storage device 830, or memory on processor 810.

The high speed controller 840 manages bandwidth-intensive operations for the electronic device 800, while the low speed controller 860 manages lower bandwidth-intensive operations. Such allocation of duties is exemplary only. In some implementations, the high-speed controller 840 is coupled to the memory 820, the display 880 (e.g., through a graphics processor or accelerator), and to the high-speed expansion ports 850, which may accept various expansion cards (not shown).

The electronic device 800 may be implemented in a number of different forms, as shown in FIG. 8. For example, it may be implemented as a standard server 800a or multiple times in a group of such servers 800a, as a laptop computer 800b, as part of a rack server system 800c, as a smartphone 800d, and/or as a tablet computer 800e.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Implementations of the subject matter and the functional operations described in this specification can be implemented in digital electronic circuitry, or in computer software, firmware, or hardware, including the structures disclosed in this specification and their structural equivalents, or in combinations of one or more of them. Moreover, subject matter described in this specification can be implemented as one or more computer program products, i.e., one or more modules of computer program instructions encoded on a computer readable medium for execution by, or to control the operation of, data processing apparatus. The computer readable medium can be a machine-readable storage device, a machine-readable storage substrate, a memory device, a composition of matter affecting a machine-readable propagated signal, or a combination of one or more of them. The terms “data processing apparatus”, “computing device” and “computing processor” encompass all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as an application, program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

One or more aspects of the disclosure can be implemented in a computing system that includes a backend component, e.g., as a data server, or that includes a middleware component, e.g., an application server, or that includes a frontend component, e.g., a client computer having a graphical user interface or a Web browser through which a user can interact with an implementation of the subject matter described in this specification, or any combination of one or more such backend, middleware, or frontend components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specifics, these should not be construed as limitations on the scope of the disclosure or of what may be claimed, but rather as descriptions of features specific to particular implementations of the disclosure. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

A number of implementations have been described. Nevertheless, it will be understood that various modifications may be made without departing from the spirit and scope of the disclosure. Accordingly, other implementations are within the scope of the following claims. For example, the actions recited in the claims can be performed in a different order and still achieve desirable results.

What is claimed is:

1. A system comprising:
data processing hardware; and
memory hardware in communication with the data processing hardware, the memory hardware storing instructions that when executed on the data processing hardware cause the data processing hardware to perform operations comprising:
receiving a plurality of input data from one or more data collection devices;
converting each of the plurality of input data to sub-data;
converting the sub-data into a fuzzy domain by using fuzzy membership functions to create fuzzy data;
performing fuzzy inference operations on the fuzzy data using a fuzzy rule base;
performing deffuzification operations to convert the fuzzy data into crisp data;
determining a heart disease diagnosis for a patient based on the crisp data; and
transmitting data indicating a dosage amount of a medication to an administration device associated with the patient based on the heart disease diagnosis, the medication configured to treat symptoms associated with heart disease.

2. The system of claim 1, wherein the operations further comprise displaying the heart disease diagnosis on a display of a healthcare provider device associated with a healthcare provider supervising the patient.

3. The system of claim 2, wherein the healthcare provider device is configured to be operated by the healthcare provider to modify the plurality of input data.

4. The system of claim 1, wherein the one or more data collection devices includes one or more of a Holter monitor, a blood pressure monitor, a cholesterol meter or monitor, a blood glucose meter or monitor, an electrocardiograph (ECG or EKG) monitor or machine, a heart rate monitor, an exercise or activity monitor, a gamma camera, a fluoroscope, a scale, an X-ray machine, a spirometer, a pulse oximeter, a capnography monitor, or a blood test machine.

5. The system of claim 1, wherein the crisp data includes a numerical value indicating on a scale the likelihood that the patient has heart disease.

6. The system of claim 1, wherein the input data includes one or more of chest pain, blood pressure (systolic, diastolic), cholesterol, blood sugar, resting electrocardiography, maximum heart rate, exercise, old peak, sex, thallium scan, age, slope (slope of peak exercise ST segment), color vessels (number of major vessels colored by fluoroscopy), body mass index (BMI), narrow vessels, FEV1/FVC ratio (Tiffeneau-Pinelli index), blood oxygen level, respiratory status, or c-reactive protein (CRP).

7. The system of claim 1, wherein the deffuzification operations include one or more of a Weighted Average Formulate (WAF) Method, a Quality Method (QM), a Maximum-Weighted Average Formula (MAX-WAF) Method, or a Center of Sums (COS) Method.

8. A method comprising:
receiving, via data processing hardware, a plurality of input data from one or more data collection devices;
converting, via the data processing hardware, each of the plurality of input data to sub-data;
converting, via the data processing hardware, the sub-data into a fuzzy domain by using fuzzy membership functions to create fuzzy data;
performing, via the data processing hardware, fuzzy inference operations on the fuzzy data using a fuzzy rule base;
performing, via the data processing hardware, deffuzification operations to convert the fuzzy data into crisp data;
determining, via the data processing hardware, a heart disease diagnosis for a patient based on the crisp data; and
transmitting, via the data processing hardware, data indicating a dosage amount of a medication to an administration device associated with the patient based on the heart disease diagnosis, the medication configured to treat symptoms associated with heart disease.

9. The method of claim 8, further comprising displaying, via the data processing hardware, the heart disease diagnosis on a display of a healthcare provider device associated with a healthcare provider supervising the patient.

10. The method of claim 9, wherein the healthcare provider device is configured to be operated by the healthcare provider to modify the plurality of input data.

11. The method of claim 8, wherein the one or more data collection devices includes one or more of a Holter monitor, a blood pressure monitor, a cholesterol meter or monitor, a blood glucose meter or monitor, an electrocardiograph (ECG or EKG) monitor or machine, a heart rate monitor, an exercise or activity monitor, a gamma camera, a fluoroscope, a scale, an X-ray machine, a spirometer, a pulse oximeter, a capnography monitor, or a blood test machine.

12. The method of claim 8, wherein the crisp data includes a numerical value indicating on a scale the likelihood that the patient has heart disease.

13. The method of claim 8, wherein the input data includes one or more of chest pain, blood pressure (systolic, diastolic), cholesterol, blood sugar, resting electrocardiography, maximum heart rate, exercise, old peak, sex, thallium scan, age, slope (slope of peak exercise ST segment), color vessels (number of major vessels colored by fluoroscopy), body mass index (BMI), narrow vessels, FEV1/FVC ratio (Tiffeneau-Pinelli index), blood oxygen level, respiratory status, or c-reactive protein (CRP).

14. The method of claim 8, wherein the deffuzification operations include one or more of a Weighted Average Formulate (WAF) Method, a Quality Method (QM), a Maximum-Weighted Average Formula (MAX-WAF) Method, or a Center of Sums (COS) Method.

15. A system comprising:
one or more data collection devices;
data processing hardware; and
memory hardware in communication with the data processing hardware, the memory hardware storing instructions that when executed on the data processing hardware cause the data processing hardware to perform operations comprising:
receiving a plurality of input data from the one or more data collection devices;
converting each of the plurality of input data to sub-data;
converting the sub-data into a fuzzy domain by using fuzzy membership functions to create fuzzy data;
performing fuzzy inference operations on the fuzzy data using a fuzzy rule base;
performing deffuzification operations to convert the fuzzy data into crisp data;
determining a heart disease diagnosis for a patient based on the crisp data; and
transmitting data indicating a dosage amount of a medication to an administration device associated with the patient based on the heart disease diagnosis, the medication configured to treat symptoms associated with heart disease.

16. The system of claim 15, wherein the operations further comprise displaying the heart disease diagnosis on a display of a healthcare provider device associated with a healthcare provider supervising the patient.

17. The system of claim 16, wherein the healthcare provider device is configured to be operated by the healthcare provider to modify the plurality of input data.

18. The system of claim 15, wherein the one or more data collection devices includes one or more of a Holter monitor, a blood pressure monitor, a cholesterol meter or monitor, a blood glucose meter or monitor, an electrocardiograph (ECG or EKG) monitor or machine, a heart rate monitor, an exercise or activity monitor, a gamma camera, a fluoroscope, a scale, an X-ray machine, a spirometer, a pulse oximeter, a capnography monitor, or a blood test machine.

19. The system of claim 15, wherein the crisp data includes a numerical value indicating on a scale the likelihood that the patient has heart disease.

20. The system of claim 15, wherein the input data includes one or more of chest pain, blood pressure (systolic, diastolic), cholesterol, blood sugar, resting electrocardiography, maximum heart rate, exercise, old peak, sex, thallium scan, age, slope (slope of peak exercise ST segment), color vessels (number of major vessels colored by fluoroscopy), body mass index (BMI), narrow vessels, FEV1/FVC ratio (Tiffeneau-Pinelli index), blood oxygen level, respiratory status, or c-reactive protein (CRP).
